(19) United States
(12) Patent Application Publication
(10) Pub. No.: US 2025/0290099 A1
(43) Pub. Date: Sep. 18, 2025

(54) MATERIALS AND METHODS FOR TARGETED GENETIC MANIPULATIONS IN CELLS

(71) Applicant: The Regents of the University of California, Oakland, CA (US)

(72) Inventors: Brian Shy, San Francisco, CA (US); Vivasvan Vykunta, San Francisco, CA (US); Alexander Marson, San Francisco, CA (US)

(73) Assignee: The Regents of the University of California, Oakland, CA (US)

(21) Appl. No.: 18/688,882

(22) PCT Filed: Aug. 30, 2022
(86) PCT No.: PCT/US2022/042004

§ 371 Date: Mar. 4, 2024

Related U.S. Application Data
(60) Provisional application No. 63/240,306, filed on Sep. 2, 2021.

(51) Int. Cl. C12N 15/90; C12N 9/22; C12N 13/00; C12N 15/11

(52) U.S. Cl. CPC C12N 15/907; C12N 9/226; C12N 13/00; C12N 15/11; C12N 2310/20

(57) ABSTRACT

Methods for editing the genome of cells such as T cells and hematopoietic stem cells are disclosed. The methods include inserting a nucleic acid sequence of an exogenous partial open reading frame (ORF) of an autosomal dominant gene (e.g., CTLA4) into an intronic target region of an endogenous autosomal dominant gene in the cell, wherein the endogenous autosomal dominant gene comprises one or more disease-causing mutations; the exogenous partial ORF of the autosomal dominant gene is free of disease-causing mutations; and insertion of the exogenous partial ORF of the autosomal dominant gene into the intronic target region results in a modified autosomal dominant gene that encodes a protein which is free of disease-causing mutations. Methods for treating haploinsufficiency and methods for increasing gene editing efficiency are also described.

### CROSS-REFERENCE TO RELATED PATENT APPLICATIONS

The present application is a 371 U.S. Patent Application of International Application No. PCT/US2022/042004 filed Aug. 30, 2022 which claims benefit of priority to U.S. Provisional Patent Application No. 63/240,306, filed Sep. 2, 2021, which are incorporated by reference for all purposes.

### STATEMENT AS TO RIGHTS TO INVENTIONS MADE UNDER FEDERALLY SPONSORED RESEARCH AND DEVELOPMENT

This invention was made with government support under Contract No. U19AI135990 awarded by The National Institutes of Health. The government has certain rights in the invention.

### REFERENCE TO A “SEQUENCE LISTING”

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Oct. 13, 2022, is entitled “081906-1338596-243710PC_SL_ST26.xml”, and is 892,570 bytes in size. The Sequence Listing is hereby incorporated by reference in its entirety for all purposes.

### BACKGROUND OF THE INVENTION

Disease-causing mutations in the cytotoxic T-lymphocyte-associated protein 4 (CTLA4) gene most commonly manifest as a Primary Immunodeficiency (PID) referred to as CTLA4 HaploInsufficiency (CHAI) or CTLA deficiency. Patients with this disease develop a variety of autoimmune manifestations and have increased susceptibility to infection. They are currently treated predominantly with IV or subcutaneous injections of a recombinant CTLA4-Ig fusion protein called abatacept. Most patients with this disease have one good copy of the affected gene, and disrupting this allele could worsen their disease.

### BRIEF SUMMARY OF THE INVENTION

Provided herein are methods for editing the genome of a cell such as a human T cell. The methods include inserting a nucleic acid sequence of an exogenous partial open reading frame (ORF) of an autosomal dominant gene into an intronic target region of an endogenous autosomal dominant gene in the T cell, wherein:
the endogenous autosomal dominant gene comprises one or more disease-causing mutations,
the exogenous partial ORF of the autosomal dominant gene is free of disease-causing mutations, and
insertion of the exogenous partial ORF of the autosomal dominant gene into the intronic target region results in a modified autosomal dominant gene that encodes a protein which is free of disease-causing mutations.

In some embodiments, the autosomal dominant gene is CTLA4, and wherein insertion of an exogenous CLTA4 partial ORF into the intronic target region of an endogenous CLTA4 gene results in a modified CTLA4 gene that encodes a CTLA4 protein which is free of disease-causing mutations. In some embodiments, the autosomal dominant gene is CD7, LRBA, CD40LG, MAGT1, WASP, RHOH, BCL10, ITGB2, IL10RA, SAP, PI3KCD, CD4, CD5, STAT3, ZAP70, PI3KR1, STIM1, CD45, CD3D, CD2, CD3G, CD3Z, or CD3E, and wherein insertion of an exogenous CD7, LRBA, CD40LG, MAGT1, WASP, RHOH, BCL10, ITGB2, IL10RA, SAP, PI3KCD, CD4, CD5, STAT3, ZAP70, PI3KR1, STIM1, CD45, CD3D, CD2, CD3G, CD3Z, or CD3E partial ORF into the intronic target region of an endogenous CD7, LRBA, CD40LG, MAGT1, WASP, RHOH, BCL10, ITGB2, IL10RA, SAP, PI3KCD, CD4, CD5, STAT3, ZAP70, PI3KR1, STIM1, CD45, CD3D, CD2, CD3G, CD3Z, or CD3E gene results in a modified CD7, LRBA, CD40LG, MAGT1, WASP, RHOH, BCL10, ITGB2, IL10RA, SAP, PI3KCD, CD4, CD5, STAT3, ZAP70, PI3KR1, STIM1, CD45, CD3D, CD2, CD3G, CD3Z, or CD3Eene that encodes a CD7, LRBA, CD40LG, MAGT1, WASP, RHOH, BCL10, ITGB2, IL10RA, SAP, PI3KCD, CD4, CD5, STAT3, ZAP70, PI3KR1, STIM1, CD45, CD3D, CD2, CD3G, CD3Z, or CD3E protein which is free of disease-causing mutations In some embodiments, the intronic target region is in intron 1 of the endogenous CTLA4 gene and the exogenous CTLA4 partial ORF comprises exons 2-4 of CTLA4. In some embodiments, the intronic target region is in intron 1 of the endogenous CTLA4 gene and the exogenous CTLA4 partial ORF consists of exons 2-4 of CTLA4.

In some embodiments, the nucleic acid sequence of the exogenous CTLA4, CD7, LRBA, CD40LG, MAGT1, WASP, RHOH, BCL10, ITGB2, IL10RA, SAP, PI3KCD, CD4, CD5, STAT3, ZAP70, PI3KR1, STIM1, CD45, CD3D, CD2, CD3G, CD3Z, or CD3E partial ORF is inserted into the intronic target region by introducing into the cell: (a) a targeted nuclease that creates an insertion site in the intronic target region; (b) a guide RNA that specifically hybridizes to the intronic target region; and (c) a DNA template comprising the nucleic acid sequence of the exogenous CTLA4, CD7, LRBA, CD40LG, MAGT1, WASP, RHOH, BCL10, ITGB2, IL10RA, SAP, PI3KCD, CD4, CD5, STAT3, ZAP70, PI3KR1, STIM1, CD45, CD3D, CD2, CD3G, CD3Z, or CD3E partial ORF.

In some embodiments, the DNA template is a single-stranded DNA template, the 5′ end and the 3′ end of the DNA template comprise nucleotide sequences that are homologous to genomic sequences flanking the intronic target region, the DNA template further comprises a nuclease binding sequence, wherein the nuclease binding sequence forms a double-stranded duplex with a complementary nucleotide sequence. In some embodiments, the double-stranded duplex is formed with an oligonucleotide or polynucleotide comprising the complementary nucleotide sequence. In some embodiments, the targeted nuclease is a Cas9 nuclease. In some embodiments, the targeted nuclease, the guide RNA, and the DNA template are introduced into the cell as a ribonucleoprotein complex (RNP)-DNA template complex. In some embodiments, introducing the RNP-DNA template complex into the cell comprises electroporation.

In some embodiments, the targeted nuclease, the guide RNA, and the DNA template are introduced into the cell in the presence of one or more small molecules selected from the group consisting of a DNA-dependent protein kinase (DNA-PK) inhibitor, a histone deacetylase (HDAC) inhibitor, and a cell division cycle 7-related protein kinase (CDC7) inhibitor. In some embodiments, the DNA-PK inhibitor is (S)-(2-chloro-4-fluoro-5-(7-morpholinoquinazolin-4-yl)phenyl)(6-methoxypyridazin-3-yl)methanol (M3814) or 8-(dibenzo[b,d]thiophen-4-yl)-2-morpholino-4H-chromen-4-one (NU7441). In some embodiments, the HDAC inhibitor is [R-(E,E)]-7-[4-(dimethylamino)phenyl]-N-hydroxy-4,6-dimethyl-7-oxo-2,4-heptadienamide (trichostatin A). In some embodiments, the CDC7 inhibitor is (S)-8-chloro-2-(pyrrolidin-2-yl)benzofuro[3,2-d]pyrimidin-4(3H)-one hydrochloride (XL413).

In some embodiments, the method further comprises administering the cell comprising the modified autosomal dominant gene to a human subject. In some embodiments, the subject is the same subject from whom the cell having the endogenous autosomal dominant gene was obtained. In some embodiments, the cell is a human cell. In some embodiments, the cell is a T cell or a hematopoietic stem cell.

Also provided are isolated cells having an edited genome, which cell is prepared according to the method described above or elsewhere herein.

Also provided is an isolated cell having an edited genome comprising a modified CTLA4 gene comprising an CTLA4 open reading frame (ORF) comprising an endogenous exon 1 and exogenous exons 2-4, wherein the exogenous exons are free of disease-causing mutations. In some embodiments, the cell is a human cell. In some embodiments, the cell is a T cell or a hematopoietic stem cell.

Also provided is a method for treating a haploinsufficiency. In some embodiments, the method comprises administering a therapeutically effective amount of cells, as described above or elsewhere herein, to a subject in need thereof. In some embodiments, the haploinsufficiency causes a primary immunodeficiency. In some embodiments, the haploinsufficiency is CTLA4, CD7, LRBA, CD40LG, MAGT1, WASP, RHOH, BCL10, ITGB2, IL10RA, SAP, PI3KCD, CD4, CD5, STAT3, ZAP70, PI3KR1, STIM1, CD45, CD3D, CD2, CD3G, CD3Z, or CD3E haploinsufficiency.

Also provided is a method for generating nucleotide deletions in a target gene in a cell, the method comprising: electroporating the cell in the presence of:
(i) a ribonucleoprotein (RNP) complex comprising a targeted nuclease and a guide RNA, wherein the guide RNA specifically hybridizes to a nucleotide sequence in the target gene, and
(ii) one or more small molecules selected from the group consisting of a DNA-dependent protein kinase (DNA-PK) inhibitor, a histone deacetylase (HDAC) inhibitor, and a cell division cycle 7-related protein kinase (CDC7) inhibitor,
wherein electroporating the cell is conducted in the absence of a homology directed repair template, thereby introducing the RNP into the cell; and
maintaining the cell under conditions for forming one or more nucleotide deletions in the target gene.

Also provided is a method for modifying a target gene in a cell, the method comprising:
electroporating the cell in the presence of:
(a) a ribonucleoprotein (RNP) complex comprising a guide RNA and a targeted nuclease, wherein the guide RNA specifically hybridizes to a nucleotide sequence in a genomic target region and the targeted nuclease creates an insertion site in the genomic target region;
(b) a single-stranded DNA template comprising an exogenous nucleic acid sequence, wherein the 5′ end and the 3′ end of the DNA template comprise nucleotide sequences that are homologous to genomic sequences flanking the genomic target region, and wherein the DNA template further comprises a nuclease binding sequence, wherein the nuclease binding sequence forms a double-stranded duplex with a complementary nucleotide sequence; and
(c) one or more molecules selected from the group consisting of a DNA-dependent protein kinase (DNA-PK) inhibitor, a histone deacetylase (HDAC) inhibitor, and a cell division cycle 7-related protein kinase (CDC7) inhibitor,
thereby modifying the target gene.

In some embodiments, the DNA-PK inhibitor is (S)-(2-chloro-4-fluoro-5-(7-morpholinoquinazolin-4-yl)phenyl)(6-methoxypyridazin-3-yl)methanol (M3814) or 8-(dibenzo[b,d]thiophen-4-yl)-2-morpholino-4H-chromen-4-one (NU7441). In some embodiments, the HDAC inhibitor is [R-(E,E)]-7-[4-(dimethylamino)phenyl]-N-hydroxy-4,6-dimethyl-7-oxo-2,4-heptadienamide (trichostatin A). In some embodiments, the CDC7 inhibitor is (S)-8-chloro-2-(pyrrolidin-2-yl)benzofuro[3,2-d]pyrimidin-4(3H)-one hydrochloride (XL413). Also provided is a method for modifying a target gene in a cell, the method comprising: combining the cell with:
(a) a targeted nuclease that creates an insertion site in a genomic target region in the cell,
(b) a guide RNA that specifically hybridizes to the genomic target region,
(c) a DNA template comprising an exogenous nucleic acid sequence, and
(d) one or more small molecules selected from the group consisting of
(S)-(2-chloro-4-fluoro-5-(7-morpholinoquinazolin-4-yl)phenyl)(6-methoxypyridazin-3-yl)methanol (M3814) at a concentration of 0.2 μM to 1 μM,
8-(dibenzo[b,d]thiophen-4-yl)-2-morpholino-4H-chromen-4-one (NU7441) at a concentration of 0.2 μM to 1 μM,
[R-(E,E)]-7-[4-(dimethylamino)phenyl]-N-hydroxy-4,6-dimethyl-7-oxo-2,4-heptadienamide (trichostatin A) at a concentration of 0.015 μM to 0.075 μM, and
(S)-8-chloro-2-(pyrrolidin-2-yl)benzofuro[3,2-d]pyrimidin-4(3H)-one hydrochloride (XL413) at a concentration of 2 μM to 15 μM;
electroporating the cell, the targeted nuclease, the guide RNA, the DNA template, and the small molecules; and
maintaining the cell under conditions for insertion of the exogenous nucleic acid sequence into the insertion site, thereby modifying the target gene.

In some embodiments, the cell is combined with the M3814 and trichostatin A. In some embodiments, the cell is combined with the M3814, the trichostatin A, and the XL413In some embodiments, the cell is a human cell. In some embodiments, the human cell is a T cell or a hematopoietic stem cell.

Also provided herein are methods for treating a haploinsufficiency. The methods include administering a therapeutically effective amount of modified cells as described herein to a subject in need thereof.

Also provided herein are methods of gene editing having increased efficiency. The methods include electroporating cells (e.g., T cells) in the presence of a ribonucleoprotein (RNP) complex comprising a guide RNA and a targeted nuclease; a DNA template; and one or more molecules selected from the group consisting of a DNA-dependent protein kinase (DNA-PK) inhibitor, a histone deacetylase (HDAC) inhibitor, and a cell division cycle 7-related protein kinase (CDC7) inhibitor.

### BRIEF DESCRIPTION OF THE DRAWINGS

FIG. 1 shows the development of ssCTS templates for high yield knock-in. (a) Diagram of hybrid ssDNA HDRT designs incorporating CTS sites (ssCTS). (b) Panel of ssCTS designs tested. (c) Knock-in efficiency for each ssCTS design using a CD5-HA knock-in construct at 160 nM-4 uM concentration assessed by flow cytometry. Dotted line represents mean knock-in percentage for control ssDNA HDRTs without CTS (construct a, grey). (d-f) Knock-in strategy, gating, knock-in efficiency, live cell counts, and knock-in cell counts are shown for large ssCTS templates including (d) a tNGFR knock-in at the IL2RA locus, (e) a IL2RA-GFP fusion protein knock-in to the IL2RA locus, or (f) two different HDRTs inserting a BCMA-CAR construct at TRAC locus via two different gRNAs (g526 and g527). Each experiment was performed with T cells from 2 independent healthy human blood donors. Error bars indicate standard deviation. RNP=Ribonucleoprotein, CTS=Cas9 Target Site, ssCTS=ssDNA HDRT+CTS sites, HDRT=homology-directed-repair template.

FIG. 2 shows screening with short CD5-HA ssCTS templates. (a) Diagram of CD5-HA knock-in strategy and control ssDNA HDRTs. (b) Representative flow cytometry plots demonstrating CD5-HA knock-in. (c) Live cell counts for each ssCTS design using a CD5-HA knock-in construct at 160 nM-4 uM concentration. Each experiment was performed with T cells from 2 independent healthy human blood donors. Error bars indicate standard deviation. RNP=Ribonucleoprotein, HDRT=homology-directed-repair template.

FIG. 3 shows optimization of ssCTS design with large CD5-HA HDRTs. (a-f) Comparison of different CTS designs with a large ˜2.7 kb CD5-HA knock-in construct. (a) Diagram of long CD5-HA knock-in strategy, representative flow cytometry plot, % knock-in, live cell counts, and knock-in cell yield counts. (b) Comparison of CTS with a gRNA target sequence that is specific for the cognate RNP (+CD5 CTS), an alternative gRNA sequence (+IL2RA CTS), a CTS incorporating a PAM site and scrambled gRNA sequence (+scramble CTS), or an equivalent amount of dsDNA within the 5′ end of the homology arm (+end protection). (c) Comparison of complementary oligos covering different regions of the CTS and surrounding sequences. Constructs with CTS sites on both 5′ and 3′ end (green bars), 5′ end only (blue blurs), or 3′ end only (red bars) are shown on the right panel. (d) Evaluation of varied 5′ ends including different length of buffer sequence upstream of the CTS site. *indicates no data available for the marked column. (e) Comparison of CTS with different numbers of scrambled bases at the 5′ end of the gRNA target sequence using WT or SpyFi Cas9. (f) Length of homology arm that is covered by the complementary oligonucleotide. (g) Comparison of HDRT variations for knock-in constructs targeting a tNGFR marker across 22 different target loci. Shown for each construct are live cell counts, knock-in cell count yields, relative % knock-in and relative knock-in counts compared to dsDNA templates. Each experiment was performed with T cells from 2 independent healthy human blood donors. Error bars indicate standard deviation. CTS=Cas9 Target Site, PAM=Protospacer Adjacent Motif, HDRT=homology-directed-repair template

FIG. 4 shows evaluation of ssCTS design considerations. (a-e) Comparison of different CTS designs with an IL2RA-GFP knock-in construct targeting IL2RA locus assessed by flow cytometry. (a) Comparison of CTS with a gRNA target sequence that is specific for the cognate RNP (+IL2RA CTS), an alternative gRNA sequence (+CD5 CTS), a CTS incorporating a PAM site and scrambled gRNA sequence (+scramble CTS), or an equivalent amount of dsDNA within the 5′ end of the homology arm (+end protection). (b) Comparison of complementary oligos covering varying regions of the CTS and surrounding sequences (design schematics left; knock-in results right). Constructs with CTS sites on both 5′ and 3′ end (green bars), 5′ end only (blue blurs), or 3′ end only (red bars) are shown on the right panel with two best performing designs indicated (right). (c) Evaluation of varied 5′ ends including different length of buffer sequence upstream of the CTS site. (d) Comparison of CTS designs with varying numbers of scrambled bases at the 5′ end of the gRNA target sequence using WT or SpyFi Cas9. (e) Knock-in percentages are shown with varying length of homology arm covered by the complementary oligonucleotide. Each experiment was performed with T cells from 2 independent healthy donors. Error bars indicate standard deviation. All comparisons except for panel b include complementary oligos covering the entire 5′ Buffer+gRNA+PAM+homology arms. CTS=Cas9 Target Site, PAM=Protospacer Adjacent Motif, HDRT=homology-directed-repair template, HA=homology arms.

FIG. 5 shows the application of ssCTS knock-in templates across diverse target loci, knock-in constructs, and primary human hematopoietic cell types. (a) Knock-in efficiencies for constructs targeting a tNGFR marker to 22 different target genome loci. (b-d) Comparison of large ssDNA and dsDNA HDRTs with CTS sites for knock-in of a pooled library of 2.6-3.6 kb polycistronic constructs targeted to the TRAC locus. Shown for each HDRT variation is (b) relative % knock-in in comparison to maximum for dsDNA+CTS templates, (c) relative knock-in cell count yields in comparison to maximum for dsDNA+CTS templates, and (d) representation of each library member in knock-in cells post-electroporation in comparison to construct representation the input plasmid pool. (e-g) Comparison of knock-in cell yields using ssDNA (red) and dsDNA HDRTs (blue) with CTS sites across a variety of primary human hematopoietic cell types. Note, different cell type comparisons are performed with different blood donors. All comparisons were performed using a knock-in construct generating an CLTA-mCherry fusion at the CLTA locus. Shown for each cell type using HDRT concentrations from 5-160 nM are (e) knock-in cell count yields, (f) maximum fold-change in knock-in count yields (relative to dsCTS templates), and (g) maximum % knock-in. (h) Evaluation of ssCTS templates +/−M3814+TSA (MT) or M3814+TSA+XL413 (MTX) inhibitor combinations with a panel of 44 different knock-in constructs targeting a tNGFR marker across 22 different target loci including genes implicated in Primary Immunodeficiencies (PID) or with potential importance for T cell engineering. 2 gRNAs and corresponding ssCTS templates were used for each gene (g1 and g2). All experiments were performed with T cells from 2 independent healthy donors. Error bars indicate standard deviation. CTS=Cas9 Target Site, HDRT=homology-directed-repair template, tNGFR=truncated Nerve Growth Factor Receptor, dsCTS=dsDNA HDRT+CTS sites, ssCTS=ssDNA HDRT+CTS sites, kb=kilobase, MT=M3814+TSA, MTX=M3814+TSA+XL413.

FIG. 6 shows the evaluation of small molecule inhibitors to boost knock-in in primary human T cells. (a) Evaluation of relative increase in % knock-in using an ssDNA CD5-HA knock-in construct over varied concentrations of 5 different small molecule inhibitors assessed by flow cytometry. Red bars indicate concentrations chosen for subsequent experiments. (b) Comparison of relative % knock-in (top) and live cell counts (bottom) with small molecule inhibitor combinations. Combinations chosen for subsequent experiments are highlighted in blue (MT) and yellow (MTX). (c) Comparison of dsCTS and ssCTS templates in combination with small molecule inhibitors for 5 different knock-in constructs including a large CD5-HA HDRT (˜2.7 kb), a tNGFR knock-in to the IL2RA gene (˜1.5 kb), an mCherry fusion in the Clathrin gene (˜1.5 kb), a near full length CTLA4-GFP fusion to the CTLA4 gene (˜2.1 kb), and a full length IL2RA-GFP fusion to the IL2RA gene (˜2.3 kb). (d) Evaluation of live cell counts with MT and MTX inhibitor combinations using 44 different knock-in constructs targeting a tNGFR marker across 22 different target loci with 2 gRNA per gene (g1 and g2). Panel a, b, and d were each performed with T cells from 2 independent healthy human blood donors. Panel c was performed with T cells from 4-6 independent healthy human blood donors. Error bars indicate standard deviation. CTS=Cas9 Target Site, HDRT=homology-directed-repair template, dsCTS=dsDNA+CTS HDRT, ssCTS=ssDNA+CTS HDRT, TSA=Trichostatin A, HDR Enhancer=IDT Alt-R HDR Enhancer, MT=M3814+TSA, MTX=M3814+TSA+XL413.

FIG. 7 shows IL2RA and CTLA4 ORF replacement strategies. (a) Gating for GFP+ cells is shown with WT and S166N IL2RA-GFP knock-in constructs. (b) Diagram of the CTLA4 gene (top), CTLA4 protein levels (bottom), and cutting efficiency (bottom) illustrating a screening panel of 12 gRNAs examined within exon 1 and intron 1. gRNAs were assessed in activated CD4+ T cells for protein disruption by CTLA4 flow cytometric analysis (flow plots and top row of numbers demonstrate the % of CTLA4-negative cells for each donor), and for cutting efficiency as determined by TIDE indel analysis (see Brinkman, E. K. et al. Nucleic acids research 46, e58, doi:10.1093/nar/gky164 (2018). The bottom row of numbers indicate the % indel at target locus. (c) CTLA4 expression levels assessed by flow cytometry with endogenous protein (black) and WT CTLA4-GFP knock-in protein (red) are shown for CD4− T cells, CD4+ T cells, and regulatory T cells with (dotted line) and without (solid line) stimulation. (d) Gating for GFPhi cells is shown for WT, R70W, R75W, and T124P CTLA4-GFP knock-in cells. Each experiment was performed with T cells from 2 independent healthy human blood donors. Error bars indicate standard deviation. WT=Wild-Type, Treg=regulatory T cell.

FIG. 8 shows whole open reading frame (ORF) replacement at target genes for therapeutic and diagnostic human T cell editing. (a-d) IL2RA exon 1-8 ORF replacement strategy. (a) Diagram of the IL2RA gene with reported patient coding mutations and knock-in strategy using an IL2RA-GFP fusion protein targeted to exon 1. The S166N mutation examined explored in panel c-d is noted. (b) TL2RA and GFP expression in CD4+ T cells electroporated with IL2RA-GFP ssCTS templates and cognate RNP followed by MTX inhibitor combination (green), in comparison to RNP only (red), or no electroporation control cells (blue). (c) Comparison of extracellular (surface staining) and intracellular (staining in permeabilized cells which includes total surface and intracellular protein) IL2RA expression with WT and S166N IL2RA-GFP knock-ins assessed by flow cytometry. Percent IL2RA+ is shown for each panel. (d) Localization of WT and S166N IL2RA-GFP protein by fluorescence microscopy. (e-i) CTLA4 exon 2-4 ORF replacement strategy. (e) Diagram of the CTLA4 gene with reported patient mutations and knock-in strategy using a CTLA4-GFP fusion protein targeted to intron 1. The R70W, R75W, T124P mutations examined in panel g-i are noted. (f) CTLA4 and GFP expression in CD4+ T cells electroporated with CTLA4-GFP ssCTS templates and cognate RNP followed by MTX inhibitor combination (green), in comparison to RNP only (red), or no electroporation control cells (blue). (g) Quantification of percent knock-in for WT, R70W, R75W, and T124P constructs electroporated with ssCTS templates and treated with the MTX inhibitor combination assess by flow cytometry. (h) Structure of CTLA4 dimer with CD80/86 interaction domain highlighted (yellow) along with location of R70W (blue), R75W (orange), and T124P (green) mutations. (i) Comparison of extracellular CTLA4 (surface staining), intracellular CTLA4 (staining in permeabilized cells which includes total surface and intracellular protein), and biotinylated recombinant CD80 ligand interaction stained with Streptavidin-APC in WT, R70W, R75W, and T124P knock-in CD4+ T cells. Each experiment was performed with T cells from 2 independent healthy human blood donors. Error bars indicate standard deviation. RNP=Cas9 Ribonucleoprotein, HDRT=homology-directed-repair template, DAPI=4′,6-diamidino-2-phenylindole nuclear stain, rCD80=recombinant CD80.

FIG. 9 shows a GMP-compatible process for non-viral CAR-T cell manufacturing. (a) Diagram of non-viral CAR-T cell manufacturing process. T cells are isolated from peripheral blood and activated on Day 0 with anti-CD3/anti-CD28 Dynabeads, IL-7, and IL-15. Cells are electroporated using the Maxcyte GTx electroporator on Day 2 with Cas9 RNPs+ ssCTS HDRTs and then expanded for an additional 7-10 days using G-Rex 100M culture vessels supplemented with IL-7+IL-15. (b) Representative Day 10 flow plots showing BCMA-CAR knock-in for Control (No inhibitors), M3814, and M3814+TSA (MT) conditions. (c) BCMA-CAR knock-in rates on Day 7 and Day 10 for each condition. (d) Absolute number of CAR+ cells on Day 7 and Day 10. Dotted line shows an estimated patient dose of ˜100×10⁶ CAR+ T cells. (e) T cell immunophenotype on Day 10 based on CD45RA and CD62L expression. (f) In vitro killing of BCMA+ MM1S multiple myeloma cell lines in comparison to unmodified T cells from same blood donors. Each experiment was performed with T cells from 2 independent healthy human blood donors. Error bars indicate standard deviation. Panel a was generated in part using graphics created by Biorender.com. RNP=Ribonucleoprotein, CTS=Cas9 Target Site, ssCTS=ssDNA HDRT+CTS sites, HDRT=homology-directed-repair template, M=M3814, MT=M3814+TSA, Tscm=T stem cell memory, Tcm=T central memory, Tm=T effector memory, Teff=T effector.

FIG. 10 shows non-viral CAR-T development with GMP-compatible reagents and equipment. (a) Comparison of Genscript HDRTs with internally generated HDRTs for both ssCTS (top) and dsCTS templates (bottom). Shown for each are % knock-in, live cell counts, and knock-in cell counts in comparison to internally generated ssDNA or dsDNA controls, respectively. (b) Flow plots for T cell immunophenotype analysis based on CD45RA and CD62L expression at Day 7 and Day 10 post-activation. (c) Gating strategy for extended flow cytometric analysis demonstrating CD45RA+CD62L+CD45RO-CCR7+CD95+ population immunophenotypically consistent with a Tscm population. (d) Live cell counts for large-scale GMP-compatible manufacturing process at Day 7 and Day 10 post-activation. Each experiment was performed with T cells from 2 independent healthy human blood donors. Error bars indicate standard deviation. RNP=Ribonucleoprotein, CTS=Cas9 Target Site, dsCTS=dsDNA HDRT+CTS sites, ssCTS=ssDNA HDRT+CTS sites, HDRT=homology-directed-repair template, M=M3814, MT=M3814+TSA, MTX=M3814+TSA+XL413.

FIG. 11 shows an evaluation of CD25 (IL2RA) protein knockout in T cells electroporated with RNPs with and without inhibitors M3814 and trichostatin A (TSA).

FIG. 12 shows insertion-deletion Tracking of Indels by Decomposition (TIDE) analysis in T cells electroporated with RNPs targeting IL2RA with or without M3814 and TSA.

FIG. 13 shows the sequence of amplicons analyzed by TIDE in FIG. 12. Figure discloses SEQ ID NOS 449-450, respectively, in order of appearance.

FIG. 14 shows an evaluation of T cell receptor protein knock in T cells electroporated with RNPs with and without inhibitors M3814 and trichostatin A.

FIG. 15 shows insertion-deletion Tracking of Indels by Decomposition (TIDE) analysis in T cells electroporated with RNPs targeting TRAC with or without M3814 and TSA.

FIG. 16 shows the sequence of amplicons analyzed by TIDE in FIG. 15. Figure discloses SEQ ID NOS 451-452, respectively, in order of appearance.

FIG. 17 shows the effects of small molecule combinations on homology directed repair kinetics and efficiency.

### DETAILED DESCRIPTION OF THE INVENTION

CRISPR-Cas9 offers unprecedented opportunities to modify genome sequences in primary human cells to study disease variants and reprogram cell functions for next-generation cellular therapies. CRISPR has several potential advantages over widely used retroviral vectors including: 1) site-specific transgene insertion via homology directed repair (HDR), and 2) reductions in the cost and complexity of genome modification. Despite rapid progress with ex vivo CRISPR genome engineering, many novel research and clinical applications would be enabled by methods to further improve knock-in efficiency and the absolute yield of live knock-in cells, especially with large HDR templates (HDRT). We recently reported that Cas9 target sequences (CTS) could be introduced into double-stranded DNA (dsDNA) HDRTs to improve knock-in, but yields and efficiencies were limited by toxicity at high HDRT concentrations. Here we developed a novel system that takes advantage of lower toxicity with single-stranded DNA (ssDNA). We designed hybrid ssDNA HDRTs that incorporate CTS sites and were able to boost knock-in percentages by >5-fold and live cell yields by >7-fold relative to dsDNA HDRTs with CTS. Knock-in efficiency and yield with ssCTS HDRTs were increased further with small molecule inhibitor combinations to improve HDR. We demonstrate application of these methods across a variety of target loci, knock-in constructs, and primary human cell types to reach ultra-high HDR efficiencies (>80-90%) which we use for pathogenic gene variant modeling and universal gene replacement strategies for IL2RA and CTLA4 mutations associated with mendelian immune disorders. Finally, we develop a GMP-compatible method for fully non-viral CAR-T cell manufacturing, demonstrating knock-in efficiencies of 46-62% and generating yields of >1.5×10⁹ CAR+ T cells, well above current doses for adoptive cellular therapies. Taken together, we present a comprehensive non-viral approach to model disease associated mutations and re-write targeted genome sequences to program immune cell therapies at a scale compatible with future clinical application.

### I. Methods for Genomic Editing

Provided herein are methods for editing the genome of a cell, such as a human T cell or a hematopoietic stem cell. The methods include inserting a nucleic acid sequence of an exogenous partial open reading frame (ORF) of an autosomal dominant gene into an intronic target region of an endogenous autosomal dominant gene in the cell, wherein:
the endogenous autosomal dominant gene comprises one or more disease-causing mutations,
the exogenous partial ORF of the autosomal dominant gene is free of disease-causing mutations, and
insertion of the exogenous partial ORF of the autosomal dominant gene into the intronic target region results in a modified autosomal dominant gene that encodes a protein which is free of disease-causing mutations.

In alternative embodiments, the methods include inserting a nucleic acid sequence of an exogenous partial open reading frame (ORF) of an autosomal dominant gene into an intronic target region of an endogenous autosomal dominant gene in the cell, wherein:
the endogenous autosomal dominant gene is free of mutations (e.g., disease causing mutations),
the exogenous partial ORF of the autosomal dominant gene comprises one or more mutations (e.g., disease-causing mutations), and
insertion of the exogenous partial ORF of the autosomal dominant gene into the intronic target region results in a modified autosomal dominant gene that encodes a protein which contains the mutations. Cells resulting from such methods can be used, for example, in functional screens for elucidation of disease etiology.

Examples of autosomal dominant genes include, but are not limited to, 11q23del, ACD, ACTB, ADAM17, AICDA, AIRE, APOL1, BACH2, BCL11B, C1R, CIS, C3, CARD11, CARD14, CASP10, CD46, CFB, CFH, CFHR1, CFHR2, CFHR3, CFHR4, CFHR5, CHD7, COPA, CXCR4, Del10p13-p14, ELANE, ERBB2IP, FADD, FERMT3, FNGR1, FOXN1, GATA2, GFI1, IFIH1, IKBKB, IKZF1, IL17F, IRF2BP2, IRF3, IRF4, IRF8, ITGB2, JAK1, KMT2A, KMT2D, MAD2L2, MEFV, NCSTN, NFATS, NFKB1, NFKB2, NFKBIA, NLRC4, NLRP1, NLRP3, NLRP3, NLRP12, NOD2, OAS1, PIK3CD, PIK3R1, PLCG2, POLR3A, POLR3C, POLR3F, PSEN, PSENEN, PSMB8, PSTPIP1, PTEN, RAC2, RAD51, RAD51C, RELA, RPSA, RTEL1, SAMD9, SAMD9L, SEC61A1, SEMA3E, SERPING1, SH3BP2, SLC35C1, SRP54, SRP72, STAT1, STAT3, STAT5b, STXBP2, TBK1, TBX1, TCF3, TERC, TERT, TGFBR1, TGFBR2, THBD, TIMCAM1, TINF2, TLR3, TNFAIP, TNFRSF13B, TNFRSF1A, TNFRSF6, TNFS12, TOP2B, TP53, and TRAF3. Any intronic region of such genes may be targeted for insertion in the methods of the present disclosure.

In some embodiments, the autosomal dominant gene is CTLA4, and insertion of an exogenous CLTA4 partial ORF into the intronic target region of an endogenous CLTA4 gene results in a modified CTLA4 gene that encodes a CTLA4 protein which is free of disease-causing mutations.

In some embodiments, the intronic target region is in intron 1 of the endogenous CTLA4 gene and the exogenous CTLA4 partial ORF comprises exons 2-4 of CTLA4. For example, the intronic target region may be at chr2:203,868,052-203,870,585 in hg38 genome assembly. In some embodiments, the partial ORF is inserted at chr2:203,870,312.

In some embodiments, the nucleic acid sequence of the exogenous partial ORF of the autosomal dominant gene (e.g., CTLA4) is inserted into the intronic target region by introducing into the cell: (a) a targeted nuclease that creates an insertion site in the intronic target region; (b) a guide RNA that specifically hybridizes to the intronic target region; and (c) a DNA template comprising the nucleic acid sequence of the exogenous partial ORF.

In some embodiments, the DNA template comprises a single-stranded DNA polynucelotide (also referred to as a homology directed repair template; ssHDRT) and one or more nuclease binding sequences, wherein at least one nuclease binding sequence forms a double-stranded duplex with a complementary polynucleotide sequence. The template may contain a linear or circular ssDNA. In some embodiments, the DNA template is formed from a single polynucleotide molecule. In some embodiments, the DNA template is formed from two or more polynucleotide molecules. Various template constructs may be employed including, but not limited to, a primer construct, a mixed chain construct, a half loop construct, a hairpin construct, a hairpin/primer construct, a mixed loop construct, a cap construct, and a double hairpin construct, as described in International Pat. Appl. No. PCT/US2021/022058, which is incorporated herein by reference in its entirety. In some embodiments, the DNA template comprises a double-stranded DNA polynucleotide or a viral template such as an adenovirus associated vector (AAV).

In some embodiments:
the DNA template is a single-stranded DNA template,
the 5′ end and the 3′ end of the DNA template comprise nucleotide sequences that are homologous to genomic sequences flanking the intronic target region,
the DNA template further comprises a nuclease binding sequence, wherein the nuclease binding sequence forms a double-stranded duplex with a complementary nucleotide sequence.

### A. Primer Template Constructs

In some embodiments, the double-stranded duplex is formed between the nuclease binding sequence and an oligonucleotide or polynucleotide comprising the complementary nucleotide sequence. Template constructs containing such oligonucleotides are also referred to herein as “primer constructs” or “primer template constructs.” In general, a primer construct contains a linear, single stranded DNA template and one or two double-stranded duplex regions formed from two complementary nuclease binding sequences (also referred to as DNA-binding protein target sequences or Cas9 target sequences; CTSs). In some embodiments, the donor template contains one ssHDRT and one nuclease binding sequence. In other embodiments, the donor template contains one ssHDRT and two nuclease binding sequences. Each nuclease binding sequence forms a double-stranded duplex with a complementary polynucleotide sequence, which typically does not extend into the ssHDRT sequence. In some embodiments, the template construct can contain two polynucleotide molecules, in which one polynucleotide molecule contains a template that has one ssHDRT and one nuclease binding sequence and the second polynucleotide molecule contains a complementary polynucleotide sequence. In some embodiments, the template construct can contain three polynucleotide molecules, in which the first polynucleotide molecule contains a template that has one ssHDRT and one nuclease binding sequence and each of the second and third polynucleotide molecules contains a complementary polynucleotide sequence. The nuclease binding sequence can be located at or proximal to the 5′ and/or 3′ terminus of the donor template. Exemplary ssHDRT sequences that can be used in the compositions and methods described herein are elisted under “SEQUENCES” at the end of the application, and optionally include the listed 5′, 3′ or both 5′ and 3′ CTS sequences listed below.

In some embodiments, the DNA template has the sequence:

(SEQ ID NO: 1)
CTATtgacaaacagaagaccCGGTACAGTGCATCAAGACACAGCTACTC
CTGGGTGACAGAGGTTCAGGGCCAGCTCACTAAGTAGGCAGAAGTTTTT
GACATATACTTTGAGAGATAAAGCAAGATTCTGTACCTCAACCTTCAGA
ATTTCCCCTACCACTCATTATAGTTCCGGAGCTATATAGCTCCTATCAT
TCTatcataaccttagaataccagagaacatatcatctcatctaattat
ctcttactatatgtgaaaaaaatgaaggacatgggggaagtgtgacttg
ccccaaatcacatatttcatggtagagggCTGGGCTTGGCCATGAAGGA
GCATGAGTTCACTGAGTTCCCTTTGGCTTTTCCATGCTAGCAATGCACG
TGGCCCAGCCTGCTGTGGTACTGGCCAGCAGCCGAGGCATCGCCAGCTT
TGTGTGTGAGTATGCATCTCCAGGCAAAGCCACTGAGGTCCGGGTGACA
GTGCTTCGGCAGGCTGACAGCCAGGTGACTGAAGTCTGTGCGGCAACCT
ACATGATGGGGAATGAGTTGACCTTCCTAGATGATTCCATCTGCACGGG
CACCTCCAGTGGAAATCAAGTGAACCTCACTATCCAAGGACTGAGGGCC
ATGGACACGGGACTCTACATCTGCAAGGTGGAGCTCATGTACCCACCGC
CATACTACCTGGGCATAGGCAACGGAACCCAGATTTATGTAATTGATCC
AGAACCGTGCCCAGATTCTGACTTCCTCCTCTGGATCCTTGCAGCAGTT
AGTTCGGGGTTGTTTTTTTATAGCTTTCTCCTCACAGCTGTTTCTTTGA
GCAAAATGCTAAAGAAAAGAAGCCCTCTTACAACAGGGGTCTATGTGAA
AATGCCCCCAACAGAGCCAGAATGTGAAAAGCAATTTCAGCCTTATTTT
ATTCCCATCAATGGATCTGGAGGAACTAGCGGCAGCAAGGGCGAGGAGC
TGTTCACCGGGGTGGTGCCCATCCTGGTCGAGCTGGACGGCGACGTAAA
CGGCCACAAGTTCAGCGTGCGCGGCGAGGGCGAGGGCGATGCCACCAAC
GGCAAGCTGACCCTGAAGTTCATCTGCACCACCGGCAAGCTGCCCGTGC
CCTGGCCCACCCTCGTGACCACCCTGACCTACGGCGTGCAGTGCTTCAG
CCGCTACCCCGACCACATGAAGCGCCACGACTTCTTCAAGTCCGCCATG
CCCGAAGGCTACGTCCAGGAGCGCACCATCAGCTTCAAGGACGACGGCA
CCTACAAGACCCGCGCCGAGGTGAAGTTCGAGGGCGACACCCTGGTGAA
CCGCATCGAGCTGAAGGGCATCGACTTCAAGGAGGACGGCAACATCCTG
GGGCACAAGCTGGAGTACAACTTCAACAGCCACAACGTCTATATCACCG
CCGACAAGCAGAAGAACGGCATCAAGGCCAACTTCAAGATCCGCCACAA
CGTGGAGGACGGCAGCGTGCAGCTCGCCGACCACTACCAGCAGAACACC
CCCATCGGCGACGGCCCCGTGCTGCTGCCCGACAACCACTACCTGAGCA
CCCAGTCCGTGCTGAGCAAAGACCCCAACGAGAAGCGCGATCACATGGT
CCTgCTGGAgTTCGTGACCGCCGCCGGGATCACTTGAcaccgggtcttc
aacttgtttattgcagcttataatggttacaaataaagcaatagcatca
caaatttcacaaataaagcatttttttcactgcattctagttgtggttt
gtccaaactcatcaatgtatcttatcatgtctggaagacctgtttacct
cttctgtttgtcatatcagtgttcttcctgccacaaccatcttgAAGAA
TCTATTTCTCAGTAAGAAAATATCTTTATGGAGAGTAGCTGGAAAACAG
TTGAGAGATGGAGGGGAGGCTGGGGGTGTGGAGAGGGGAAGGGGTAAGT
GATAGATTCGTTGAAGGGGGGAGAAAAGGCCGTGGGGATGAAGCTAGAA
GGCAGAAGGGCTCCGggtcttctgtttgtcaATAG.

In some embodiments, the primer construct contains one or both of the following complementary nuclease binding sequences:

(SEQ ID NO: 2; CTLA4 ssCTS left primer)
CTGTGTCTTGATGCACTGTACCGGGTCTTCTGTTTGTCAATAG
(SEQ ID NO: 3; CTLA4 ssCTS right primer)
CTATtgacaaacagaagaccCGGAGCCCTTCTGCCTTCTAG.

In some embodiments, the DNA template further includes a protospacer adjacent motif (PAM) sequence. The Cas9 protein identifies the target nucleic acid by first identifying a 3-base pair PAM located 3′ of the target nucleic acid. Once the PAM is identified, the target gRNA in the RNP complex hybridizes to the target nucleic acid upstream of the PAM. The DNA template can further contain one or more edge sequences at either or both of the 5′ and 3′ termini of the template. An edge sequence in the donor template can facilitate binding between the donor template and the DNA-binding protein (e.g., an RNA-guided nuclease). In some embodiments, an edge sequence can have at least 2 nucleotides, e.g., between 2 and 24 nucleotides (e.g., between 2 and 22, between 2 and 20, between 2 and 18, between 2 and 16, between 2 and 14, between 2 and 12, between 2 and 10, between 2 and 8, between 2 and 6, between 2 and 4, between 4 and 24, between 6 and 24, between 8 and 24, between 10 and 24, between 12 and 24, between 14 and 24, between 16 and 24, between 18 and 24, between 20 and 24, or between 22 and 24 nucleotides; 2, 3, 4, 5, 6, 7, 8, 9, 10, 11, 12, 13, 14, 15, 16, 17, 18, 19, 20, 21, 22, 23, or 24 nucleotides).

In some embodiments, the size or length of the donor template is greater than about 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 1 kb, 1.1 kb, 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb, any size of template in between these sizes, or greater than 10 kb. For example, the size of the template can be about 200 bp to about 500 bp, about 200 bp to about 750 bp, about 200 bp to about 1 kb, about 200 bp to about 1.5 kb, about 200 bp to about 2.0 kb, about 200 bp to about 2.5 kb, about 200 bp to about 3.0 kb, about 200 bp to about 3.5 kb, about 200 bp to about 4.0 kb, about 200 bp to about 4.5 kb, about 200 bp to about 5.0 kb.

### B. Guide RNA

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence. In some embodiments, the guide sequence for targeting of CTLA-4 is GATATGACAAACAGAAGACC (SEQ ID NO:4). The guide sequence can be used in a single-guide RNA (sgRNA) as described below, or in a split crRNA+tracrRNA construct. In some embodiments, the tracrRNA sequence is AACAGCAUAGCAAGUUAAAAUAAGGCUAGUCCGUUAUCAACUUGAAAAAGUGGC ACCGAGUCGGUGCUUUUUUU (SEQ ID NO:5). In some embodiments, the gRNA comprises one of the sequences listed in Table 3 or Table A, targeting the gene designated in the Tables for each gRNA.

In some embodiments, the targeted nuclease (e.g., a Cas protein) is guided to its target DNA by a single-guide RNA (sgRNA). An sgRNA is a version of the naturally occurring two-piece guide RNA (crRNA and tracrRNA) engineered into a single, continuous sequence. An sgRNA typically contains (1) a guide sequence (e.g., the crRNA equivalent portion of the sgRNA) that targets the Cas protein to the target DNA, and (2) a scaffold sequence that interacts with a nuclease such as a Cas protein (e.g., the tracrRNAs equivalent portion of the sgRNA). An sgRNA may be selected using a software. As a non-limiting example, considerations for selecting an sgRNA can include, e.g., the PAM sequence for the Cas9 protein to be used, and strategies for minimizing off-target modifications. Tools such as NUPACK® and the CRISPR Design Tool can provide sequences for preparing the sgRNA, for assessing target modification efficiency, and/or assessing cleavage at off-target sites.

The guide sequence in the sgRNA may be complementary to a specific sequence within a target DNA. The 3′ end of the target DNA sequence can be followed by a PAM sequence. Approximately 20 nucleotides upstream of the PAM sequence is the target DNA. In general, a Cas9 protein or a variant thereof cleaves about three nucleotides upstream of the PAM sequence. The guide sequence in the sgRNA can be complementary to either strand of the target DNA.

In some embodiments, the guide sequence of an sgRNA may comprise about 10 to about 2000 nucleic acids, for example, about 10 to about 100 nucleic acids, about 10 to about 500 nucleic acids, about 10 to about 1000 nucleic acids, about 10 to about 1500 nucleic acids, about 10 to about 2000 nucleic acids, about 50 to about 100 nucleic acids, about 50 to about 500 nucleic acids, about 50 to about 1000 nucleic acids, about 50 to about 1500 nucleic acids, about 50 to about 2000 nucleic acids, about 100 to about 500 nucleic acids, about 100 to about 1000 nucleic acids, about 100 to about 1500 nucleic acids, about 100 to about 2000 nucleic acids, about 500 to about 1000 nucleic acids, about 500 to about 1500 nucleic acids, about 500 to about 2000 nucleic acids, about 1000 to about 1500 nucleic acids, about 1000 to about 2000 nucleic acids, or about 1500 to about 2000 nucleic acids at the 5′ end of the sgRNA that can direct the Cas protein to the target DNA site using RNA-DNA complementarity base pairing. In some embodiments, the guide sequence of an sgRNA comprises about 100 nucleic acids at the 5′ end of the sgRNA that can direct the Cas protein to the target DNA site using RNA-DNA complementarity base pairing. In some embodiments, the guide sequence comprises 20 nucleic acids at the 5′ end of the sgRNA that can direct the Cas protein to the target DNA site using RNA-DNA complementarity base pairing. In other embodiments, the guide sequence comprises less than 20, e.g., 19, 18, 17, 16, 15 or less, nucleic acids that are complementary to the target DNA site. In some instances, the guide sequence in the sgRNA contains at least one nucleic acid mismatch in the complementarity region of the target DNA site. In some instances, the guide sequence contains about 1 to about 10 nucleic acid mismatches in the complementarity region of the target DNA site.

The scaffold sequence in the sgRNA may serve as a protein-binding sequence that interacts with the Cas protein or a variant thereof. In some embodiments, the scaffold sequence in the sgRNA can comprise two complementary stretches of nucleotides that hybridize to one another to form a double-stranded RNA duplex (dsRNA duplex). The scaffold sequence may have structures such as lower stem, bulge, upper stem, nexus, and/or hairpin. In some embodiments, the scaffold sequence in the sgRNA can be between about 90 nucleic acids to about 120 nucleic acids, e.g., about 90 nucleic acids to about 115 nucleic acids, about 90 nucleic acids to about 110 nucleic acids, about 90 nucleic acids to about 105 nucleic acids, about 90 nucleic acids to about 100 nucleic acids, about 90 nucleic acids to about 95 nucleic acids, about 95 nucleic acids to about 120 nucleic acids, about 100 nucleic acids to about 120 nucleic acids, about 105 nucleic acids to about 120 nucleic acids, about 110 nucleic acids to about 120 nucleic acids, or about 115 nucleic acids to about 120 nucleic acids.

### C. Targetable Nuclease

As described above, in some embodiments of the compositions and methods described herein, the targetable nuclease is an RNA-guided nuclease (e.g., a Cas protein). The targetable nuclease can recognize a sequence of a target nucleic acid (e.g., a target gene within a genome), bind to the target nucleic acid, and modify the target nucleic acid. In other embodiments, the targetable nuclease can be a fusion protein that includes a protein that can bind to the target nucleic acid and a protein that can modify the target nucleic acid (e.g., a nuclease, a transcription activator or repressor).

In some embodiments, the targetable nuclease has nuclease activity. For example, the targetable nuclease can modify the target nucleic acid by cleaving the target nucleic acid. The cleaved target nucleic acid can then undergo homologous recombination with a nearby a homology directed repair (HDR) template. For example, the Cas nuclease can direct cleavage of one or both strands at a location in a target nucleic acid. Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, Cas9, and Cfp1. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref Seq. No. WP_011681470.

Cas nucleases, e.g., Cas9 nucleases, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

Cas9 protein refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 can be a fusion protein, e.g., the two catalytic domains are derived from different bacteria species.

In some embodiments, a Cas protein can be a Cas protein variant. For example, useful variants of the Cas9 nuclease can include a single inactive catalytic domain, such as a RuvC- or HNH⁻ enzyme or a nickase. A Cas9 nickase has only one active functional domain and can cut only one strand of the target DNA, thereby creating a single strand break or nick. In some embodiments, the Cas9 nuclease can be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, a Cas protein variant that lacks cleavage (e.g., nickase) activity. A Cas protein variant may contain one or more point mutations that eliminates the protein's nickase activity. In some embodiments, such Cas protein variants can be fused to other proteins and serve as targeting domains to direct the other proteins to the target nucleic acid. For example, Cas protein variants without nickase activity may be fused to transcriptional activation or repression domains to control gene expression (Ma et al., Protein and Cell, 2(11):879-888, 2011; Maeder et al., Nature Methods, 10:977-979, 2013; and Konermann et al., Nature, 517:583-588, 2014). A Cas protein variant that lacks nickase activity may be used to target genomic regions, resulting in RNA-directed transcriptional control. In some embodiments, a Cas protein variant without any cleavage (e.g., nickase) activity may be used to target an exogenous protein to the target nucleic acid. An exogenous protein may be fused to the Cas protein variant and the fusion protein may be enhanced by the addition of the anionic polymer. An exogenous protein may be an effector protein domain. An exogenous protein may be a transcription activator or repressor. Other examples of exogenous proteins include, but are not limited to, VP64-p65-Rta (VPR), VP64, P65, Krab, Ten-eleven translocation methylcytosine dioxygenase (TET), and DNA methyltransferase (DNMT). Specific Cas protein variants that lack cleavage (e.g., nickase) activity are also described below.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9(1.0)), and SpCas9 (K848A/K1003A/R1060A) (also referred to as eSpCas9(1.1)) variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

In some embodiments, a targetable nuclease can also be a fusion protein that contains a protein that can bind to the target nucleic acid and a protein that can cleave the target nucleic acid. For example, a protein that can recognize and bind to the target nucleic acid can be a Cas protein variant without any cleavage activity. A Cas protein variant without any cleavage activity can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983, or D986, as well as a mutation at H840 or N863. In some instances, the dCas9 enzyme can contain a D10A or D10N mutation. Also, the dCas9 enzyme can contain a H840A, H840Y, or H840N. In some embodiments, the dCas9 enzyme can contain D10A and H840A; D10A and H840Y; D10A and H840N; DION and H840A; DION and H840Y; or DION and H840N substitutions. The substitutions can be conservative or non-conservative substitutions to render the Cas9 polypeptide catalytically inactive and able to bind to target DNA.

In other embodiments, a protein that can recognize and bind to the target nucleic acid can be a transcription activator-like (TAL) effector DNA-binding protein or a zinc finger DNA-binding protein. The TAL effector DNA-binding protein has a central domain of DNA-binding tandem repeats usually containing 33-35 amino acids in length and two hypervariable amino acid residues at positions 12 and 13 that can recognize one or more specific DNA base pairs. The zinc finger DNA-binding protein has a DNA-binding motif that is often characterized by the absence or presence one or more zinc ions in order to coordinate and stabilize the motif fold. The zinc finger DNA-binding protein contains multiple finger-like protrusions that make tandem contacts with their target molecule. Some zinc finger DNA-binding proteins also form salt bridges to stabilize the finger-like folds. They were first identified as a DNA-binding motif in transcription factor TFIIIA from Xenopus laevis (African clawed frog), however they are now recognized to bind DNA, RNA, protein, and/or lipid substrates.

In some embodiments, a targetable nuclease in the compositions and methods described herein can be a fusion protein containing a TAL effector DNA-binding protein and a protein that can cleave the target nucleic acid (also referred to as “Transcription activator-like effector nucleases (TALEN)”). In other embodiments, a targetable nuclease in the compositions and methods described herein can be a fusion protein containing a zinc finger DNA-binding protein and a protein that can cleave the target nucleic acid. For example, a protein that can cleave the target nucleic acid can be a wild-type or mutated FokI endonuclease or the catalytic domain of FokI. Detailed descriptions of TALENs and their uses for gene editing are found, e.g., in U.S. Pat. Nos. 8,440,431; 8,440,432; 8,450,471; 8,586,363; and 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49-55. Examples of a zinc finger DNA-binding protein fused to a protein that can cleave the target nucleic acid are described in the art and include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

In some embodiments, the targetable nuclease does not have nuclease activity. For example, the targetable nuclease (e.g., a targetable nuclease without any nuclease activity) can regulate the expression of the target nucleic acid. In some embodiments, the targetable nuclease can be a fusion protein that includes a protein that can bind to the target nucleic acid, such as a Cas protein variant without any cleavage activity (e.g., a dCas9), a TAL effector DNA-binding protein, and a zinc finger DNA-binding protein as described above, and a protein that can modify the target nucleic acid, such as a transcription activator or repressor.

The targetable nuclease can also be fused with a localization peptide or protein. For example, the targetable nuclease can be fused with one or more nuclear localization signal (NLS) sequences, which can direct the targetable nuclease and the RNP complexes it forms to the nucleus to modify the target nucleic acid. Examples of NLS sequences are known in the art, e.g., as described in Lange et al., J Biol Chem. 282(8):5101-5, 2007, and also include, but are not limited to, AVKRPAATKKAGQAKKKKLD (SEQ ID NO: 6), MSRRRKANPTKLSENAKKLAKEVEN (SEQ ID NO: 7), PAAKRVKLD (SEQ ID NO:8), KLKIKRPVK (SEQ ID NO:9), and PKKKRKV (SEQ ID NO:10). Examples of other peptide or proteins that can be used to a targetable nuclease, such as cell-penetrating peptides and cell-targeting peptides are available in the art and described, e.g., Vives et al., Biochim Biophys Acta. 1786(2):126-38, 2008. In some embodiments, the targeted nuclease is a Cas9 nuclease.

In some embodiments, the targeted nuclease, the guide RNA, and the DNA template are introduced into the cell as a ribonucleoprotein complex (RNP)-DNA template complex. The RNP-DNA template complex may be formed, for example, by incubating the RNP with the DNA template for less than about one minute to about thirty minutes, at a temperature of about 20° C. to about 25° C. In some embodiments, the RNP-DNA template complex and the cell are mixed prior to introducing the RNP-DNA template complex into the cell.

In some embodiments, introducing the RNP-DNA template complex into the cell comprises electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842. Additional or alternative methods, compositions, and devices for electroporating cells to introduce a RNP-DNA template complex can include those described in Geng, T. et al., J. Control Release 144, 91-100 (2010); and Wang, J., et al. Lab. Chip 10, 2057-2061 (2010).

In some embodiments, the methods further include administering a cell comprising a modified gene (e.g., an autosomal dominant gene) to a human or other subject. In some embodiments, the subject is same subject from whom the cell having the endogenous autosomal dominant gene was obtained.

### D. Genome Edited Cells

Also provided herein are isolated cells (including, but not limited to, human T cells and hematopoietic stem cells) having an edited genome. The cells can be prepared according to the methods described above. For example, isolated human T cells having an edited genome comprising a modified CTLA4 gene are provided, wherein the CTLA4 gene includes an CTLA4 open reading frame (ORF) comprising an endogenous exon 1 and exogenous exons 2-4, wherein the exogenous exons are free of disease-causing mutations.

Cell populations according to the present disclosure (e.g., a population of T cells) can be a heterogeneous population of cells and/or a heterogeneous population of different cell types. The population of cells can be heterogeneous with respect to the percentage of cells that are genomically edited. A population of cells can have greater than 10%, greater than 20%, greater than 30%, greater than 40%, greater than 50%, greater than 60%, greater than 70%, greater than 80%, or greater than 90% of the population comprise an integrated nucleotide sequence. In a certain aspect, a populations of cells comprises an integrated nucleotide sequence, wherein the integrated nucleotide sequence comprises at least a portion of a gene, the integrated nucleotide sequence is integrated at an endogenous genomic target locus, and the integrated nucleotide sequence is orientated such that the at least a portion of the gene is capable of being expressed, wherein the population of cells is substantially free of viral-mediated delivery components, and wherein greater than 10%, greater than 20%, greater than 30%, greater than 40%, greater than 50%, greater than 60%, greater than 70%, greater than 80%, or greater than 90% of the cells in the population comprise the integrated nucleotide sequence.

In some embodiments, the cell is a primary cell that is selected from the group consisting of an immune cell (e.g., a primary T cell), a blood cell, a progenitor or stem cell thereof, a mesenchymal cell, and a combination thereof. In some instances, the immune cell is selected from the group consisting of a T cell, a B cell, a dendritic cell, a natural killer cell, a macrophage, a neutrophil, an eosinophil, a basophil, a mast cell, a precursor thereof, and a combination thereof. The progenitor or stem cell can be selected from the group consisting of a hematopoietic progenitor cell, a hematopoietic stem cell, and a combination thereof. In some cases, the blood cell is a blood stem cell. In some instances, the mesenchymal cell is selected from the group consisting of a mesenchymal stem cell, a mesenchymal progenitor cell, a mesenchymal precursor cell, a differentiated mesenchymal cell, and a combination thereof. The differentiated mesenchymal cell can be selected from the group consisting of a bone cell, a cartilage cell, a muscle cell, an adipose cell, a stromal cell, a fibroblast, a dermal cell, and a combination thereof. In some embodiments, the primary cell can comprise a population of primary cells. In some cases, the population of primary cells comprises a heterogeneous population of primary cells. In other cases, the population of primary cells comprises a homogeneous population of primary cells.

Methods for modifying a target nucleic acid in a cell described herein comprise introducing into the cell a composition described herein, wherein the HDRT is integrated into the target nucleic acid. In some cases, the cells are removed from a subject, modified using any of the methods described herein and administered to the subject. In other cases, a composition described herein can be delivered to the subject in vivo. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017). A DNA template or RNP complex described herein can be introduced into cells using available methods and techniques in the art. Non-limiting examples of suitable methods include electroporation, particle gun technology, and direct microinjection. In some embodiments, the step of introducing the composition described herein into the cell comprises electroporating the composition into the cell.

### E. Small Molecules

In some embodiments, the targeted nuclease, the guide RNA, and the DNA template are introduced into the cell in the presence of one or more small molecules selected from the group consisting of a DNA-dependent protein kinase (DNA-PK) inhibitor, a histone deacetylase (HDAC) inhibitor, and a cell division cycle 7-related protein kinase (CDC7) inhibitor.

In some embodiments, the DNA-PK inhibitor is (S)-(2-chloro-4-fluoro-5-(7-morpholinoquinazolin-4-yl)phenyl)(6-methoxypyridazin-3-yl)methanol (M3814) or 8-(dibenzo[b,d]thiophen-4-yl)-2-morpholino-4H-chromen-4-one (NU7441).

In some embodiments, the HDAC inhibitor is [R-(E,E)]-7-[4-(dimethylamino)phenyl]-N-hydroxy-4,6-dimethyl-7-oxo-2,4-heptadienamide (trichostatin A).

In some embodiments, the CDC7 inhibitor is (S)-8-chloro-2-(pyrrolidin-2-yl)benzofuro[3,2-d]pyrimidin-4(3H)-one hydrochloride (XL413).

### II. Methods for Treating Haploinsufficiency

Also provided herein are methods for treating a haploinsufficiency. The methods include administering a therapeutically effective amount of cells (e.g., human T cells) as described herein to a subject in need thereof.

In some embodiments, the haploinsufficiency causes a primary immunodeficiency. The primary immunodeficiency may, for example, affect cellular and humoral immunity (e.g., as in the case of RelA haploinsufficiency and IKAROS deficiency). The immunodeficiency may be predominantly an antibody deficiency (e.g., as in the case of nuclear factor KB subunit 1 (NFKB1) deficiency) or a disease of immune dysregulation (e.g., as in the case of CTLA-4 haploinsufficiency and NFATS haploinsufficiency). The immunodeficiency may involve defects in phagocyte number or function (e.g., as in the case of 3-actin deficiency and leukocyte adhesion deficiency type 1) or defects in intrinsic and innate immunity (e.g., as in the case of trypanosomiasis and isolated congenital asplenia). The immunodeficiency may be an autoinflammatory disorder (e.g., as in the case of ADA2 deficiency and CARD14 mediated psoriasis), a complement deficiency (e.g., as in the case of Factor H deficiency or thrombomodulin deficiency), or a condition involving bone marrow failure (e.g., as in the case of ataxia pancytopenia syndrome or Fanconi anemia or SRP72 deficiency). The immunodeficiency may be a combined deficiency with associate or syndromic features (e.g., as in the case of Jacobsen syndrome and CHARGE syndrome).

In some embodiments the haploinsufficiency is due to one or more disease-causing mutations in a gene such as 11 q23del, ACD, ACTB, ADAM17, AICDA, AIRE, APOL1, BACH2, BCL11B, C1R, CIS, C3, CARD11, CARD14, CASP10, CD46, CFB, CFH, CFHR1, CFHR2, CFHR3, CFHR4, CFHR5, CHD7, COPA, CXCR4, Del10p13-p14, ELANE, ERBB2IP, FADD, FERMT3, FNGR1, FOXN1, GATA2, GFI1, IFIH1, IKBKB, IKZF1, IL17F, IRF2BP2, IRF3, IRF4, IRF8, ITGB2, JAK1, KMT2A, KMT2D, MAD2L2, MEFV, NCSTN, NFATS, NFKB1, NFKB2, NFKBIA, NLRC4, NLRP1, NLRP3, NLRP3, NLRP12, NOD2, OAS1, PIK3CD, PIK3R1, PLCG2, POLR3A, POLR3C, POLR3F, PSEN, PSENEN, PSMB8, PSTPIP1, PTEN, RAC2, RAD51, RAD51C, RELA, RPSA, RTEL1, SAMD9, SAMD9L, SEC61A1, SEMA3E, SERPING1, SH3BP2, SLC35C1, SRP54, SRP72, STAT1, STAT3, STAT5b, STXBP2, TBK1, TBX1, TCF3, TERC, TERT, TGFBR1, TGFBR2, THBD, TIMCAM1, TINF2, TLR3, TNFAIP, TNFRSF13B, TNFRSF1A, TNFRSF6, TNFS12, TOP2B, TP53, and TRAF3, or a combination thereof. In some embodiments, the haploinsufficiency is CTLA4 haploinsufficiency.

### III. Methods for Enhancing CRISPR Efficiency

Also provided herein are methods for generating nucleotide deletions in a target gene in a cell. The methods include:
electroporating the cell in the presence of:
(i) a ribonucleoprotein (RNP) complex comprising a targeted nuclease and a guide RNA, wherein the guide RNA specifically hybridizes to a nucleotide sequence in the target gene, and
(ii) one or more small molecules selected from the group consisting of a DNA-dependent protein kinase (DNA-PK) inhibitor, a histone deacetylase (HDAC) inhibitor, and a cell division cycle 7-related protein kinase (CDC7) inhibitor,
wherein electroporating the cell is conducted in the absence of a homology directed repair template, thereby introducing the RNP into the cell; and
maintaining the cell under conditions for forming one or more nucleotide deletions in the target gene.

Also provided herein are methods for modifying a target gene in a cell, comprising:
electroporating the cell in the presence of:
(a) a ribonucleoprotein (RNP) complex comprising a guide RNA and a targeted nuclease, wherein the guide RNA specifically hybridizes to a nucleotide sequence in a genomic target region and the targeted nuclease creates an insertion site in the genomic target region;
(b) a single-stranded DNA template comprising an exogenous nucleic acid sequence, wherein the 5′ end and the 3′ end of the DNA template comprise nucleotide sequences that are homologous to genomic sequences flanking the genomic target region, and wherein the DNA template further comprises a nuclease binding sequence, wherein the nuclease binding sequence forms a double-stranded duplex with a complementary nucleotide sequence; and
(c) one or more molecules selected from the group consisting of a DNA-dependent protein kinase (DNA-PK) inhibitor, a histone deacetylase (HDAC) inhibitor, and a cell division cycle 7-related protein kinase (CDC7) inhibitor,
thereby modifying the target gene.

Examples of DNA-PK inhibitors include, but are not limited to pyrazolopyrimidines (as described, for example, in WO 2020/238900), quinazoline carboxamides and other substituted quinazolines (as described, for example, in WO 2013/163190), substituted dibenzothiophenes (as described for example, in WO 2006/109081). The amount of the DNA-PK inhibitor will depend, in part, on factors such as the particular inhibitor employed, the structure of the DNA template, and the conditions under which the template is introduced into the cell. In some embodiments, the cell, the DNA template, and other components are combined with the DNA-PK inhibitor at a concentration of about 0.01 μM to about 10 μM, or about 0.05 μM to about 5 μM, or about 0.1 μM to about 2.5 μM, or about 0.2 μM to about 2.5 μM, or about 0.2 μM to about 1.5 μM, or about 0.2 μM to about 1 μM, or about 0.5 μM to about 1.5 μM, or about 0.8 μM to about 1.2 μM.

In some embodiments, the DNA-PK inhibitor is a substituted quinazoline or a substituted dibenzothiophene which may be employed, for example, at a concentration of about 0.2 μM to about 2.5 μM, or about 0.2 μM to about 1.5 μM, or about 0.2 μM to about 1 μM. In some embodiments, the DNA-PK inhibitor is (S)-(2-chloro-4-fluoro-5-(7-morpholinoquinazolin-4-yl)phenyl)(6-methoxypyridazin-3-yl)methanol (M3814) or 8-(dibenzo[b,d]thiophen-4-yl)-2-morpholino-4H-chromen-4-one (NU7441).

Examples of HDAC inhibitors include, but are not limited to hydroxamic acids (e.g., trichostatin A, vorinostat, and the like), benzamides (e.g., entinostat, tacedinaline, mocetinostat, and the like), cyclic peptides and related analogs (e.g., trapoxin, apicidin, largazole, and the like), and aliphatic acids/esters (e.g., phenylbutyrate, valproic acid, and the like). The amount of the HDAC inhibitor will depend, in part, on various factors as described above. In some embodiments, the cell, the DNA template, and other components are combined with the HDAC inhibitor at a concentration of about 0.005 μM to 0.09 μM, or about 0.01 μM to 0.08 μM, or about 0.015 μM to 0.075 μM, or about 0.02 μM to 0.075 μM, or about 0.03 μM to 0.07 μM, or about 0.04 μM to 0.06 μM.

In some embodiments, the HDAC inhibitor is a hydroxamic acid which may be employed, for example, at a concentration of about 0.01 μM to 0.08 μM, or about 0.015 μM to 0.075 μM, or about 0.02 μM to 0.075 μM. In some embodiments, the HDAC inhibitor is trichostatin A.

Examples of CDC7 inhibitors include, but are not limited to pyrimidinones (as described, for example, in WO 2018/087527 and WO 2011/102399), indazoles (as described, for example in WO 2007/124288) and pyrrolopyridines (as described, for example, in WO 2005/063746). The amount of the CDC7 inhibitor will depend, in part, on various factors as described above with respect to DNA-PK inhibitors and HDAC inhibitors. In some embodiments, the cell, the DNA template, and other components are combined with the CDC7 inhibitor at a concentration of about 0.1 μM to about 75 μM, or about 0.5 μM to about 50 μM, or about 1 μM to about 25 μM, or about 2 μM to about 25 μM, or about 2 μM to about 15 μM, or about 2 μM to about 10 μM, or about 5 μM to about 15 μM, or about 8 μM to about 12 μM.

In some embodiments, the CDC7 inhibitor is a pyrimidinone (e.g., a benzofuropyrimidinone) which may be employed, for example, at a concentration of about 2 μM to about 25 μM, or about 2 μM to about 15 μM, or about 2 μM to about 12 μM. In some embodiments, the CDC7 inhibitor is (S)-8-chloro-2-(pyrrolidin-2-yl)benzofuro[3,2-d]pyrimidin-4(3H)-one hydrochloride (XL413).

Also provided herein are methods for modifying a target gene in a cell, comprising:
combining the cell with:
(a) a targeted nuclease that creates an insertion site in a genomic target region in the cell,
(b) a guide RNA that specifically hybridizes to the genomic target region,
(c) a DNA template comprising an exogenous nucleic acid sequence, and
(d) one or more small molecules selected from the group consisting of
   (S)-(2-chloro-4-fluoro-5-(7-morpholinoquinazolin-4-yl)phenyl)(6-methoxypyridazin-3-yl)methanol (M3814) at a concentration of 0.2 μM to 1 μM,
   8-(dibenzo[b,d]thiophen-4-yl)-2-morpholino-4H-chromen-4-one (NU7441) at a concentration of 0.2 μM to 1 μM,
   [R-(E,E)]-7-[4-(dimethylamino)phenyl]-N-hydroxy-4,6-dimethyl-7-oxo-2,4-heptadienamide (trichostatin A) at a concentration of 0.015 μM to 0.075 μM, and
   (S)-8-chloro-2-(pyrrolidin-2-yl)benzofuro[3,2-d]pyrimidin-4(3H)-one hydrochloride (XL413) at a concentration of 2 μM to 15 μM;
electroporating the cell, the targeted nuclease, the guide RNA, the DNA template, and the small molecules; and
maintaining the cell under conditions for insertion of the exogenous nucleic acid sequence into the insertion site, thereby modifying the target gene.

In some embodiments, the cell is combined with the M3814 and trichostatin A. In some embodiments, the cell is combined with the M3814, the trichostatin A, and the XL413.

### IV. Examples

### Example 1. Materials and Methods

Cell Culture. Primary adult blood cells were obtained from anonymous healthy human donors as a leukapheresis pack purchased from StemCell Technologies, Inc. or Allcells Inc, or as a Trima residual from Vitalant. If needed, peripheral blood mononuclear cells were isolated by Ficoll-Paque (GE Healthcare) centrifugation. Primary human cell types were then further isolated by positive and/or negative selection using EasySep magnetic cell isolation kits purchased from StemCell for CD3+ T cells (Cat #17951), CD4+ T cells (Cat #17952), CD8+ T cells (Cat #17953), B cells (Cat #17954), NK cells (Cat #17955), or CD4+CD127lowCD25+ regulatory T cells (Cat #18063) per manufacturer instructions. Primary human γδ T cells were isolated using a custom γδ T cell negative isolation kit without CD16 and CD25 depletion obtained from StemCell. Primary adult peripheral blood G-CSF-mobilized CD34+ hematopoietic stem cells were purchased from StemExpress, LLC.

With the exception of GMP-compatible scale-up experiments (described separately below), isolated CD3+, CD4+, CD8+, and γδ T cells were activated at 1×10⁶ cells mL⁻¹ for 2 days in complete XVivo15 medium (Lonza) (5% fetal bovine serum, 50 μM 2-mercaptoethanol, 10 mM N-acetyl L-cysteine) supplemented with anti-human CD3/CD28 magnetic Dynabeads (CTS, ThermoFisher) in a 1:1 ratio with cells, 500 U mL⁻¹ of IL-2 (UCSF Pharmacy), and 5 ng mL⁻¹ of IL-7 and IL-15 (R&D Systems). Regulatory T cells were activated at 1×10⁶ cells mL⁻¹ for 2 days in complete XVivo15 supplemented with magnetic Treg Xpander CTS Dynabeads (ThermoFisher) at a 1:1 bead to cell ratio and 500 U mL⁻¹ of IL-2 (UCSF Pharmacy). Isolated B cells were activated at 1×10⁶ cells mL⁻¹ for 2 days in IMIDM medium (ThermoFisher) with 10% fetal bovine serum, 50 μM 2-mercaptoethanol, 100 ng mL-1 MEGACD40L (Enzo), 200 ng mL⁻¹ anti-human RP105 (Biolegend), 500 U mL⁻¹ IL-2 (UCSF Pharmacy), 50 ng mL⁻¹ IL-10 (ThermoFisher), and 10 ng mL⁻¹ IL-15 (R&D Systems). Isolated NK cells were activated at 1×10⁶ cells mL⁻¹ for 5 days in XVivo15 medium (Lonza) with 5% fetal bovine serum, 50 μM 2-mercaptoethanol, 10 mM N-acetyl L-cysteine, 1000 U mL⁻¹ IL-2, and MACSiBead Particles pre-coated with anti-human CD335 (NKp46) and CD2 antibodies based on manufacturer guidelines (Miltenyi Biotec). Primary adult CD34+ HSCs were cultured at 0.5×10⁶ cells per mL in SFEMII medium supplemented with CC 110 cytokine cocktail (StemCell).

For GMP-compatible scale-up experiments, CD3+ T cells were activated with anti-human CD3/CD28 magnetic Dynabeads (CTS, ThermoFisher) in a 1:1 ratio with 100 U mL⁻¹ of IL-7 and 10 U mL⁻¹ IL-15 (R&D Systems) in tissue culture flasks. Post-electroporation, cells were expanded in G-Rex 100M gas-permeable culture vessels (Wilson Wolf) supplemented with 100 U mL⁻¹ of IL-7 and 10 U mL⁻¹ IL-15 every 2-3 days for a total 7 or 10 day expansion as indicated.

RNP Formulation. For most experiments (excluding GMP-compatible scale-up described separately below), ribonucleoproteins (RNP) were produced by complexing a two-component gRNA to Cas9 with addition of either a Poly-glutamic acid (PGA) or ssDNAenh electroporation enhancer, as previously described (Nguyen, D. N. et al. Nature biotechnology 38, 44-49, (2020)). Synthetic CRISPR RNA (crRNA, with guide sequences listed in Table 1) and trans-activating crRNA (tracrRNA) were chemically synthesized (Edit-R, Dharmacon Horizon), resuspended in 10 mM Tris-HCl (pH 7.4) with 150 mM KCl or TDT duplex buffer at a concentration of 160 μM, and stored in aliquots at −80′° C. 15-50 kDa PGA was purchased from Sigma and resuspended to 100 mg ml-1 in water, sterile filtered, and stored at −80° C. prior to use. The ssDNAenh electroporation enhancer was purchased from IDT (TTAGCTCTGTTTACGTCCCAGCGGGCATGAGAGTA ACAAGAGGGTGTGGTAATATTACGGTACCGAGCACTATCGATACAATATGTGTCATA CGGACACG; SEQ ID NO: 11), resuspended to 100 μM in water, and stored at −80′° C.

TABLE 1

| | | | SEQ |
|---|---|---|---|
| | | crRNA 20 bp sequence | ID |
| crRNA ID | Target | (5′→3′) | NO: |
| G147 | CLTA | GAACGGATCCAGCTCAGCCA | 12 |
| G377 | IL2RA | TGAGCAGTCCCCACATCAGC | 13 |
| G520 | CTLA-4 | gatatgacaaacagaagacc | 4 |
| G472 | CD5 | CAGTCGCTTCCTGCCTCGGA | 14 |
| G526 | TRAC (exon) | TCAGGGTTCTGGATATCTGT | 15 |
| G527 | TRAC (intron) | CTGGATATCTGTGGGACAAG | 16 |
| CD7 g1 | CD7 | TCAGCTGCACTCGCCCGGCT | 17 |
| CD7 g2 | CD7 | CCAGAAGCAGGGGCAGCAGC | 18 |
| LRBA g1 | LRBA | CCATTGCTAGCTCACGATAA | 19 |
| LRBA g2 | LRBA | CCTTTATCGTGAGCTAGCAA | 20 |
| CD40LG g1 | CD40LG | AAAGTTGAAATGGTATCTTC | 21 |
| CD40LG g2 | CD40LG | ATGCTGTGTTAAAGTTGAAA | 22 |
| MAGT1 g1 | MAGT1 | ACGTAATGTGAGGGGTCTCC | 23 |
| MAGT1 g2 | MAGT1 | GGCCCCTTGCCTTTCCTCAT | 24 |
| WASP g1 | WASP | CTTTCTGCCCTTGTCTTCTC | 25 |
| WASP g2 | WASP | ATGAGTGGGGGCCCAATGGG | 26 |
| RHOH g1 | RHOH | GGACTTCAGAGTAGGACAGC | 27 |
| RHOH g2 | RHOH | TCAGAGTAGGACAGCAGGCT | 28 |
| BCL10 g1 | BCL10 | AGGTCCTCCTCGGTGAGGGA | 29 |
| BCL10 g2 | BCL10 | CTCGGTGAGGGACGGTGCGG | 30 |
| ITGB2 g1 | ITGB2 | AGCATGTCCTGTGGAGGGAA | 31 |
| ITGB2 g2 | ITGB2 | AGGCCCAGCATGTCCTGTGG | 32 |
| IL 10RA g1 | IL10RA | CTGGGCGCGCCGCATCGTCC | 33 |
| IL 10RA g2 | IL10RA | GCCAGCAGCACTACGAGGCA | 34 |
| SAP g1 | SAP | CCTTGCACAGTTCTCCTCCT | 35 |
| SAP g2 | SAP | CAGCCACTGCGTCCATGGCC | 36 |
| PI3KCD g1 | PI3KCD | TTTTTAGGACAACTGTCATC | 37 |
| PI3KCD g2 | PI3KCD | TAACAACGCAGGATGCCCCC | 38 |
| CD4 g1 | CD4 | TCGGCAAGGCCACAATGAAC | 39 |
| CD4 g2 | CD4 | GCTCAGGCCCCTGCCTCCCT | 40 |
| CD5 g1 | CD5 | AGCAGGTACAAGGTGGCCAG | 41 |
| CD5 g2 | CD5 | CATGGGGTCTCTGCAACCGC | 42 |
| STAT3 g1 | STAT3 | TTGGGACCCCTGATTTTAGC | 43 |
| STAT3 g2 | STAT3 | GCTGCTGTAGCTGATTCCAT | 44 |
| ZAP70 g1 | ZAP70 | TGCTGCCGTAGAAGAAGGGC | 45 |
| ZAP70 g2 | ZAP70 | GAGATGCTGCCGTAGAAGAA | 46 |
| PI3KR1 g1 | PI3KR1 | AACCAGGCTCAACTGTTGCA | 47 |
| PI3KR1 g2 | PI3KR1 | ATTTGCAAACATGAGTGCTG | 48 |
| STIM1 g1 | STIM1 | ACGCATACATCCATGACTCT | 49 |
| STIM1 g2 | STIM1 | CAGTCAACATGCGTCTGATG | 50 |
| CD45 g1 | CD45 | CACAAATACATGGTCATATC | 51 |
| CD45 g2 | CD45 | GTATTTGTGGCTTAAACTCT | 52 |
| CD3D g1 | CD3D | GAACATAGCACGTTTCTCTC | 53 |
| CD3D g2 | CD3D | GGATGAGTTCCGCTGGGAGA | 54 |
| CD2 g1 | CD2 | ACATGGAAAGCTCATCTTAG | 55 |
| CD2 g2 | CD2 | TACATGGAAAGCTCATCTTA | 56 |
| CD3G g1 | CD3G | CCATGTCAGTCTCTGTCCTC | 57 |
| CD3G g2 | CD3G | CCGGAGGACAGAGACTGACA | 58 |
| CD3Z g1 | CD3Z | AGGGAAAGGACAAGATGAAG | 59 |
| CD3Z g2 | CD3Z | GCCTCCCAGCCTCTTTCTGA | 60 |
| CD3E g1 | CD3E | AGATGCAGTCGGGCACTCAC | 61 |
| CD3E g2 | CD3E | CCATGAAACAAAGATGCAGT | 62 |

To make gRNA, aliquots of crRNA and tracrRNA were thawed, mixed 1:1 v/v, and annealed by incubation at 37° C. for 30 min to form an 80 μM gRNA solution. PGA or ssDNAenh were mixed into gRNA solutions at a 0.8:1 volume ratio prior to adding 40 μM Cas9-NLS (Berkeley QB3 MacroLab) at a 1:1 v/v to attain a molar ratio of sgRNA:Cas9 of 2:1. Final RNP mixtures were incubated at 37° C. for 15-30 minutes after a thorough mix. Based on a Cas9 protein basis, 50 pmol of RNP was used for each electroporation.

For GMP-compatible scale-up experiments, synthetic single guide RNA (sgRNA) was purchased from Synthego, resuspended to 160 μM, aliquoted and stored at −80° C. SpyFi Cas9 nuclease was purchased from Aldevron LLC, aliquoted, and stored at −20° C. For RNP formulation, aliquots of ssDNAenh and sgRNA solutions were thawed and mixed at a 0.8:1 volume ratio prior to adding SpyFi Cas9 at a 2:1 molar ratio of sgRNA:Cas9. Final RNP mixtures were incubated at 37° C. for 15-30 minutes prior to electroporation.

HDRT Template Preparation. Short ssDNA HDRTs (<200 bp) were directly synthesized (Ultramer oligonucleotides, IDT), resuspended to 100 uM in dH20, and stored at −20° C. prior to use. Long dsDNA HDRTs encoding various gene insertions and 300-600 bp homology arms were synthesized as gBlocks (IDT) and cloned into a pUC19 plasmid in-house or purchased directly from Genscript Biotech. These plasmids then served as a template for generating a PCR amplicon. CTS sites were incorporated through additional 5′ sequence added to the base PCR primers. Amplicons were generated with KAPA HiFi polymerase (Kapa Biosystems), purified by SPRI bead cleanup, and resuspended in water to 0.5-2 μg μl−1 measured by light absorbance on a NanoDrop spectrophotometer (Thermo Fisher), as previously described (Nguyen et al. supra; Roth, T. L. et al. Nature 559, 405-409, (2018)).

For most experiments requiring long ssDNA (excluding GMP-compatible scale-up described separately below), a ssDNA isolation protocol adapted from Wakimoto et al. using biotinylated primers and streptavidin-coated magnetic beads was used (Current protocols in molecular biology 107, 2 15 11-19, (2014)). Amplicons were generated as described above using primers that include a 5′ biotin modification (IDT) on either the forward or reverse PCR primer. ˜20 uL Streptavidin C1 Dynabeads (ThermoFisher, Cat #65001) per 1 picomole of amplicon were rinsed 3 times with 1× Binding & Wash (B&W) buffer (prepared at 2× concentration and stored at RT using 10 mL 1M TRIS-HCl pH 7.5, 2 mL 0.5M EDTA, 116.88 g NaCl, 1 L dH20) using magnetic separation. The washed beads and the PCR amplicon were then resuspended in B&W buffer for 30 minutes at room temperature to capture the biotinylated DNA. The mixtures were washed twice with B&W buffer after which the supernatant was removed and replaced with 0.125M NaOH Melt Solution (prepared fresh) to denature the dsDNA. The solution is placed back on the magnet for 5 minutes and the supernatant containing the non-biotinylated strand is removed gently with non-stick pipettes and mixed immediately with Neutralization Buffer (100 uL 3M Sodium Acetate pH 5.2 and 4.9 mL 1×TE Buffer, prepared fresh). Resulting ssDNA was purified and concentrated using a SPRI bead cleanup, as described previously, and quantified on a Nanodrop spectrophotometer (Thermo Fisher).

Large-Scale ssDNA Production. For GMP-compatible scale-up experiments, research grade long single-stranded DNA was manufactured at large scale by Genscript Biotech via a proprietary isothermal enzymatic reaction process (PCT/CN2019/128948). To be brief, sequence verified template on plasmid vector is first be converted into uridine modified linear dsDNA fragments via PCR amplification. The linear dsDNA is then treated with USER® Enzyme and T4 ligase (Cat. #M5505S and M0202T, New England BioLabs) to form a self-ligated dsDNA circle with nicking sites. This nick containing dsDNA circle is used as an amplification template for rolling circle amplification, which is carried out by phi29 DNA polymerase (Cat. #M0269L, New England BioLabs) in a high fidelity and linear amplification manner. The product of rolling circle amplification is ssDNA concatemers with repeats of target fragment and a palindromic adapter sequence. The annealing process is followed to let the palindromic adapter sequence form a hairpin structure, and then BspQI restriction enzyme (Cat. #R0712L, New England BioLabs) is added in the reaction system to recognize the stem part of the hairpin and digest the concatemer intermediates into target ssDNA monomers and hairpin adapters. The crude product is further purified by EndoFree® Plasmid Maxi Kit (Qiagen, Cat. #12362), to harvest the target ssDNA and remove hairpin adapters, enzymes, reaction buffer, and endotoxin residues.

For production of the 2,923nt BCMA-CAR encoding ssDNA material, amplification primers were synthesized to add specially designed adapter sequences at the 5′ and 3′ ends of the target sequence via PCR method. The uridine modified forward and reverse primer sequences manufactured by Genscript were: 5′-AACTATACUACGTCAATCGGCTCTTCACACTACTACAGTGCCAATAG-3′ (SEQ ID NO: 74) and 5′-TATAGTUACGTCAATCGGC TCTTCACACCGTCTGACTAACATAACCTG-3′ (SEQ ID NO: 75), respectively. The cycle number of the PCR reaction was set as 20, and 300 g of linear dsDNA fragment was produced and purified by QIAquick PCR Purification Kit (Qiagen, Cat. #28706). All of the purified 300 μg of linear dsDNA was treated with USER enzyme and T4 ligase to prepare the rolling circle amplification template, and then, it was used as the template for a 100 mL RCA reaction. All of the isothermal enzymatic reactions and annealing process were done on Eppendorf ThermoMixer® C. The final purified ssDNA sample was eluted with nuclease-free water (Sigma Aldrich, Cat. #W4502) from the silica column of an EndoFree® Plasmid Maxi Kit, and then passed single-use 0.22 μm sterile filter (Millipore, Cat. #SLGV033RS). Before lyophilization and final packaging, the ssDNA material was quantified by Nanodrop One^{C} (Thermo Fisher) by UV 260 nm absorbance in single-stranded DNA mode. The sequence integrity was confirmed by Sanger sequencing, and the homogeneity was measured by 2% agarose gel electrophoresis as a single band. Quality control for biosafety of the ssDNA material was also evaluated: endotoxin residue was determined as <10 EU/mg by an endotoxin test kit (Bioendo, Cat. #KC5028), protein residue level was below the minimum detection threshold of Micro BCA Protein Assay Kit (Thermo Fisher, Cat. #23235), and no bacterial colonies formed in bioburden detection.

Electroporation and use of small molecule inhibitors. Except for GMP-compatible scale-up experiments, primary cells were isolated on day 2 of culture (HSCs, CD3+, CD4+, CD8+, γδ, and regulatory T cells) or day 5 (NK cells) and electroporated using the Lonza 4D 96-well electroporation system as previously described (Nguyen, supra). CD3+, CD4+, CD8+, γδ, and regulatory T cells were debeaded using an EasySep magnet (StemCell). Immediately prior to electroporation, cells were centrifuged at 90 g for 10 minutes and then resuspended at 0.4×10⁶ HSCs, 0.5×10⁶-1.0×10⁶ T cells, 0.5×10⁶ NK cells, or 0.5×10⁶ B cells per 20 uL Lonza P3 buffer. HDRT and RNP formulations were mixed and incubated for at least 5 minutes, then combined with cells and transferred to the Lonza 96-well electroporation shuttle. B cells, NK cells, and all T cell subtypes were electroporated using pulse code EH-115 while HSCs were electroporated with pulse code ER-100. Following electroporation, cells were rescued with prewarmed growth media and incubated for at least 15 minutes. Cells were then transferred to fresh plates or flasks and diluted to 0.5-1.0×10⁶ cells mL⁻¹ in each respective growth medium as described above. Fresh cytokines and media were added every 2-3 days.

Trichostatin A (TSA) (Cayman Chemical), Nedisertib (M3814) (MedKoo Biosciences), XL413 hydrochloride (XL413) (Fisher Scientific), NU7441 (Fisher Scientific), and Alt-R HDR enhancer (IDT) were prepared and stored as aliquots per manufacturer guidelines. For experiments using small molecule inhibitors, cells were incubated with the indicated concentrations upon addition of fresh growth media following the 15 minute rescue step, and removed by media exchange after 24 hours. Longer incubation times of 48 and 72 hours did not improve knock-in efficiency further and were associated with increased toxicity (data not shown).

For GMP-compatible scale-up experiments, activated cells were separated from beads on day 2 and centrifuged for 10 minutes at 90 g. After removing the supernatant, cells were resuspended in Maxcyte Electroporation Buffer at 200×10⁶ cells mL⁻¹. HDRTs and RNPs were mixed and incubated for at least 5 minutes before being combining with cells. The mixture was then transferred to Maxcyte OC-1000 electroporation cuvettes. Cuvettes were filled up to ˜60% of the total volume (˜600 uL) and electroporated with pulse code Expanded T cell 4-2. Immediately following electroporations, ˜400 uL prewarmed XVivo15 media was added to the cuvette and cells were incubated for 15 minutes, then transferred to G-Rex culture vessels as described above.

Flow Cytometry. All flow cytometry was performed on an Attune NxT flow cytometer with a 96-well autosampler (Thermo Fisher Scientific). Unless otherwise indicated, cells were collected 3-5 days post-electroporation, resuspended in FACS buffer (1%-2% BSA in PBS) and stained with Ghost Dye red 780 (Tonbo) and the indicated cell-surface and intracellular markers (see Table 2 for antibodies). For intracellular staining, cells were stained for surface markers and then prepared for intracellular staining using True-Nuclear Transcription Factor staining kits (Biolegend). For experiments demonstrating stimulation response, cells were re-activated 24 hours prior to analysis using ImmunoCult Human CD3/CD28/CD2 T Cell Activation reagent (StemCell). Analysis was done using FlowJo v10 software. All gating strategies included exclusion of subcellular debris, singlet gating, and live:dead stain. Final graphs were produced with Prism (GraphPad), and figures were compiled with Illustrator (Adobe).

TABLE 2

| Target | Fluor | Vendor | Clone |
|---|---|---|---|
| Biotinylated Human BCMA/TNFRSF17 | — | Acro | — |
| Protein, His, Avitag | | Biosystems | |
| Biotinylated Human B7-1/CD80 Protein, | — | Acro | — |
| Fc, Avitag | | Biosystems | |
| Human TruStain FcX ™ (Fc Receptor Blocking | — | Biolegend | — |
| Solution) | | | |
| FOXP3 | Alexa 647 | Biolegend | 206D |
| Fixable Viability Dye eFluor ™ 780 | APC- | Invitrogen | — |
| | eFluor ™ | | |
| | 780 | | |
| GhostDye Red 780 | — | Tonbo | — |
| HA-Tag | Alexa 647 | Cell Signalling | 6E2 |
| Helios | PE | Biolegend | 22F6 |
| Hoechst 33342 | — | Invitrogen | — |
| LIVE/DEAD ® Fixable Aqua Dead Cell Stain | — | ThermoFisher | — |
| | | Scientific | |
| Myc-Tag | Alexa 647 | Cell Signalling | 9B11 |
| Streptavidin | PE | BD | — |
| Streptavidin | APC | Biolegend | — |
| CD19 | PacBlue | Biolegend | HIB19 |
| CD197 (CCR7) | PE | Miltenyi | REA108 |
| CTLA4 | BV-421 | BD | BNI3 |
| CD25 | BV-421 | Biolegend | BC96 |
| CD271 (NGFR) | FITC | Biolegend | ME20.4 |
| CD3 | APC | Biolegend | OKT3 |
| CD3 | PE-Cy7 | Biolegend | OKT3 |
| CD34 | BV-421 | Biolegend | 561 |
| CD4 | PE-Cy7 | Biolegend | OKT4 |
| CD4 | PE | BD | RPA-T4 |
| CD4 | FITC | Biolegend | SK3 |
| CD45RA | APC | Biolegend | HI100 |
| CD45RA | BV-650 | BD | HI100 |
| CD45RO | FITC | Tonbo | UCHL1 |
| CD5 | PacBlue | Beckman | BL1a |
| | | Coulter | |
| CD56 | BV-421 | Biolegend | 5.1H11 |
| CD62L | BV-421 | BD | DREG- |
| | | | 56 |
| CD8 | BV-421 | Biolegend | RPA-T8 |
| CD95 (Fas) | BV-711 | BioLegend | DX2 |
| TCR gamma/delta | FITC | Invitrogen | 5A6.E9 |
| TCR α/β | BV-421 | Biolegend | IP26 |

### Example 2. Development of ssCTS Templates for High-Efficiency and Low-Toxicity HDR in Primary Human T Cells

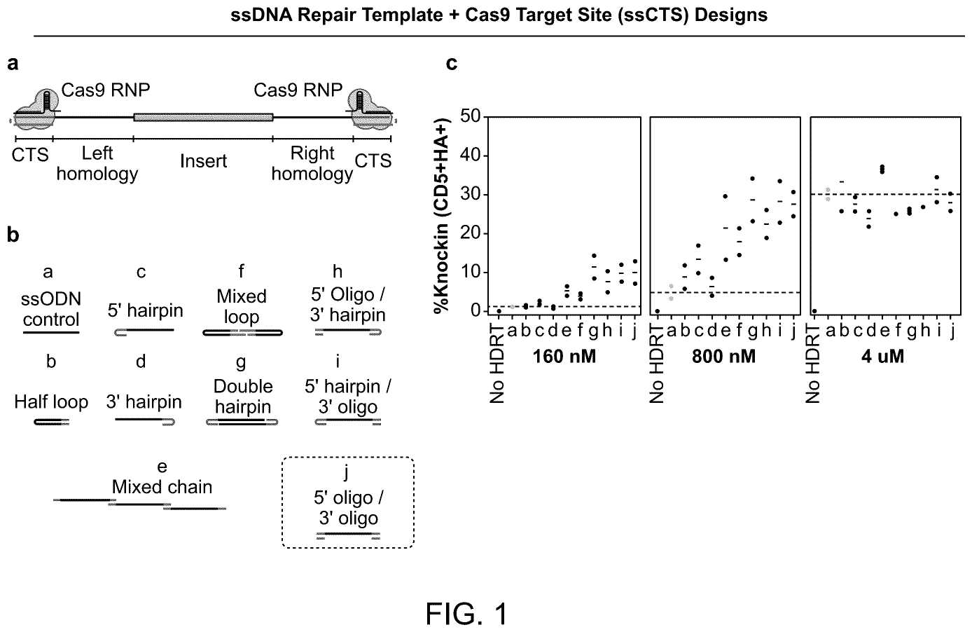

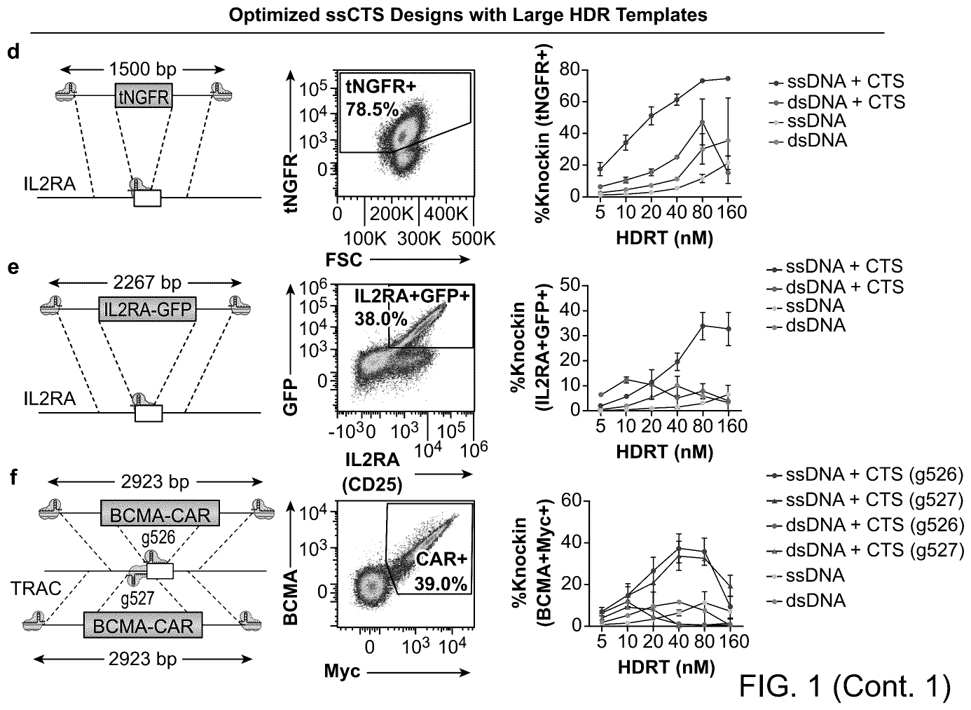

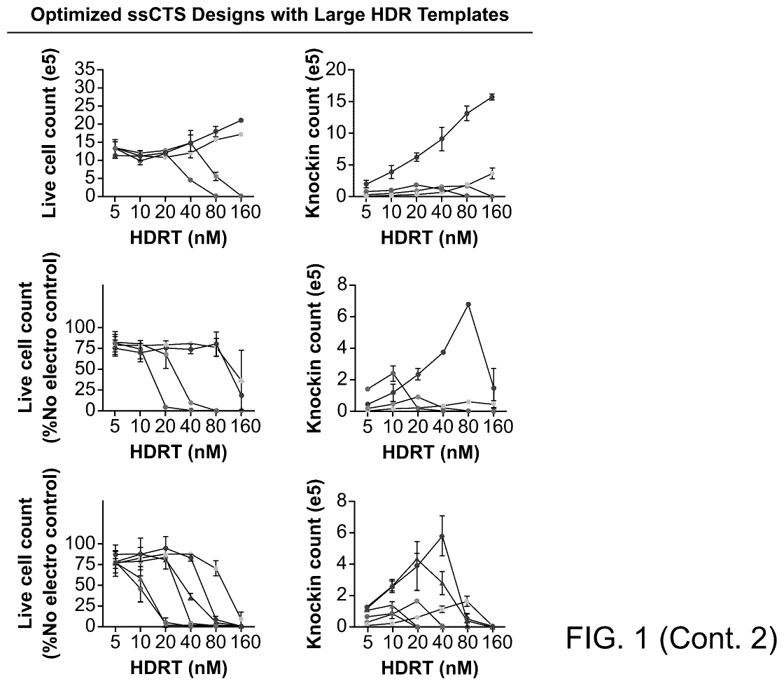

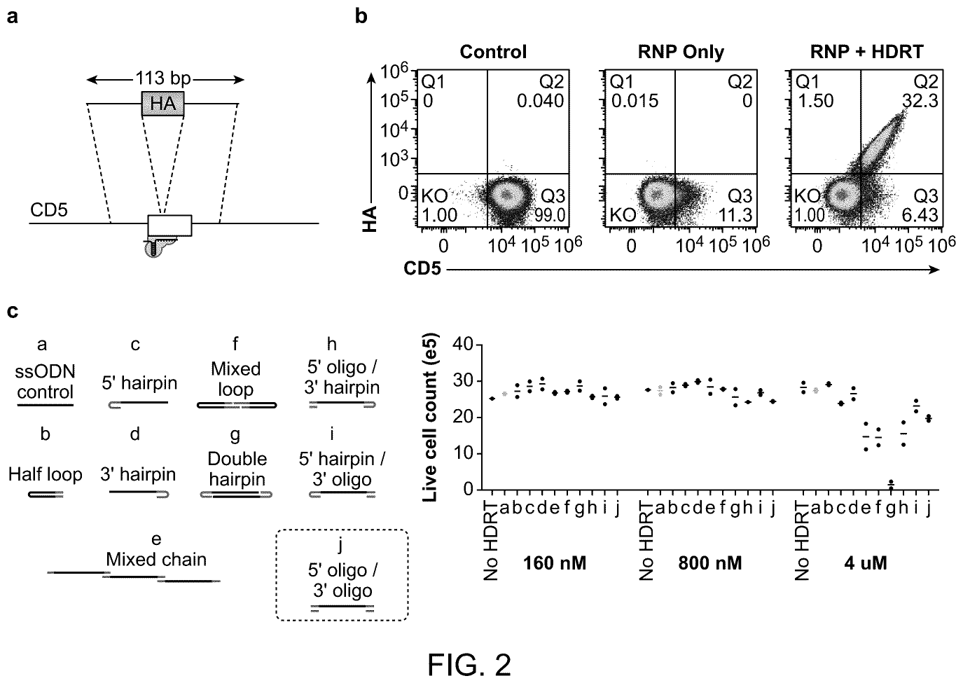

We previously developed a method to enhance delivery of dsDNA HDRTs through incorporation of Cas9 target sites (CTS) which include a gRNA target sequence and an NGG Protospacer-Adjacent-Motif (PAM) on each end of the template (Nguyen et al., supra). In comparison to dsDNA, ssDNA is associated with lower toxicity, which we reasoned could further improve knock-in efficiency and cell yield with large DNA templates if combined with CTS technology (Roth, et al., supra). We screened a variety of hybrid structures composed predominantly of ssDNA with small stretches of dsDNA incorporating the CTS sites through hairpin loops, annealed complementary oligonucleotides, or more complex secondary structures (FIG. 1A-C). We rapidly screened to compare HDRT designs using short 113-195 nt HDRTs that generate an N-terminal CD5-HA fusion protein easily detectable by flow cytometry (FIG. 2A-B). We found that the majority of these ssCTS designs increased knock-in efficiency (FIG. 1C). Improved efficiency with the ssCTS templates was apparent only at the lower 2 concentrations (160 nM and 800 nM), above which the knock-in efficiency appeared to hit a maximum of ˜30% that was achievable with unmodified ssDNA HDRTs (FIG. 1C, grey). These results suggested that ssCTS designs would be beneficial in situations where the HDRT concentration is limited, such as with large HDRTs that typically reach toxicity in the 10-320 nM range depending on their length and format.

For evaluation of large HDRTs, we chose an ssCTS design that incorporates CTS sites on both the 5′ and 3′ end via annealed complementary oligonucleotides, which are easy to design for research and clinical applications. In our panel of tested ssCTS constructs, this design demonstrated maximal enhancement of knock-in efficiency (FIG. 1B-C, “j”), low toxicity FIG. 2C), and provided the simplest process for generating CTS ends compared to hairpin loops or more complicated structures. Long ssDNA and dsDNA HDRTs ranging from 1500 nt to 2923 nt were generated with and without CTS sites (FIG. 1D-F). These templates target a knock-in detectable by flow cytometry (tNGFR, IL2RA-GFP fusion, or BCMA-CAR) to the IL2RA or TRAC locus. We evaluated post-electroporation knock-in efficiency, toxicity (based on live cell counts), and absolute yield of successful knock-in counts using primary T cells isolated from healthy human blood donors. Inclusion of CTS sites enhanced the knock-in efficiency of both dsDNA and ssDNA constructs across concentrations until toxic doses were reached, after which knock-in efficiency progressively decreased. ssCTS constructs demonstrated uniformly higher knock-in efficiencies and lower toxicity in comparison with dsCTS templates, generating up to 7-fold more knock-in cells at optimal non-toxic concentrations. The use of ssCTS templates allowed us to achieve up to 78.5% knock-in with a ˜1.5 kb tNGFR construct, or 38% for a ˜2.3 kb IL2RA-GFP construct targeting the IL2RA locus; and up to 39% knock-in with a ˜2.9 kb BCMA-specific CAR construct targeting the TRAC locus at HDRT concentrations compatible with high yields of live knock-in cells.

### Example 3. Exploration and Optimization of ssCTS Design Parameters for Large HDRTs

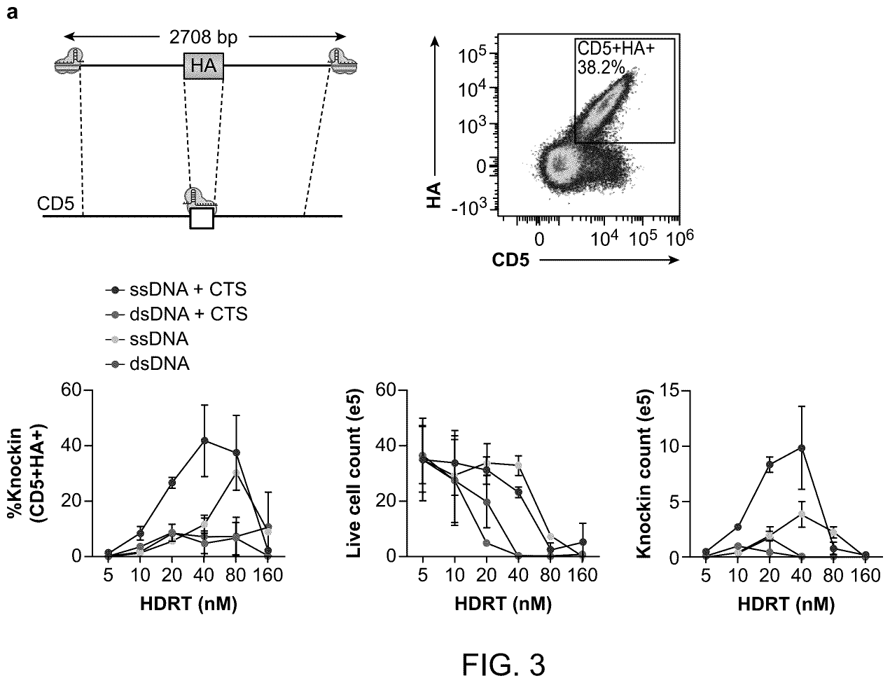

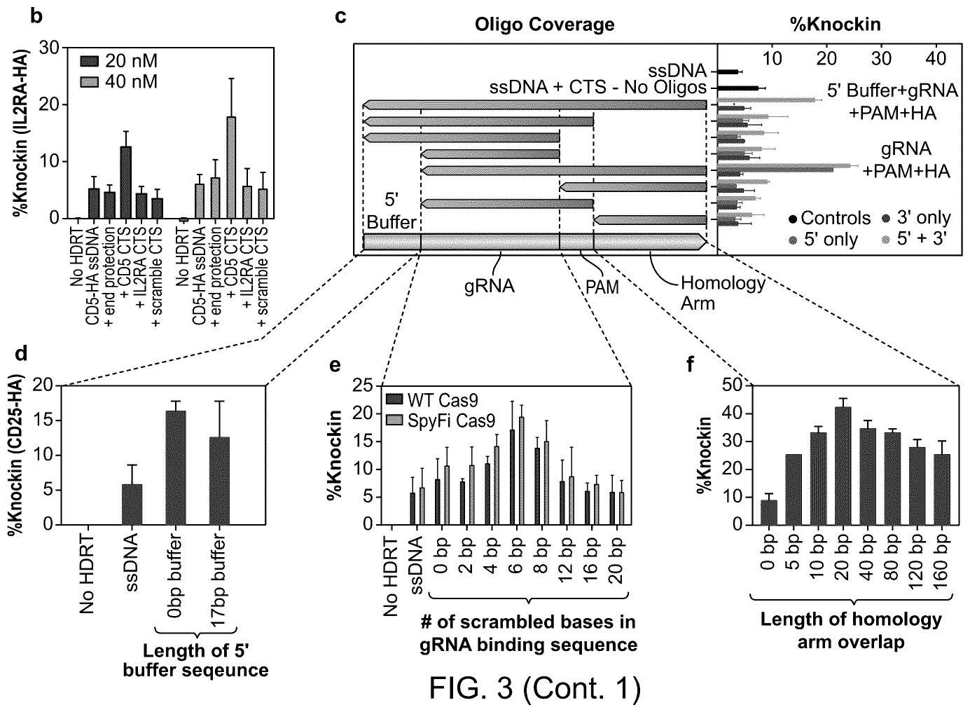

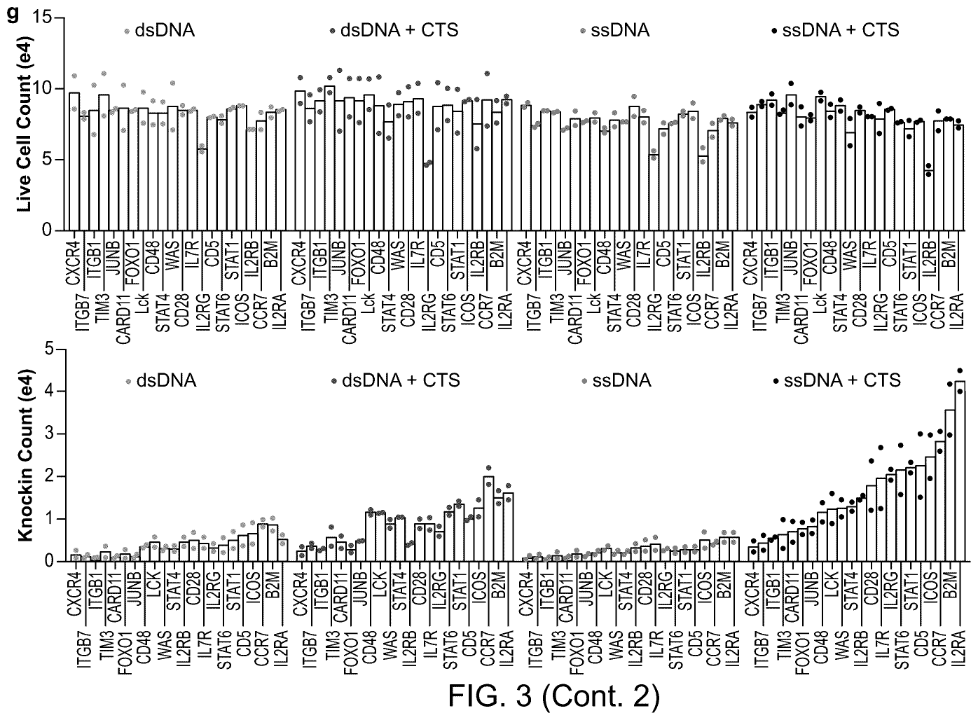

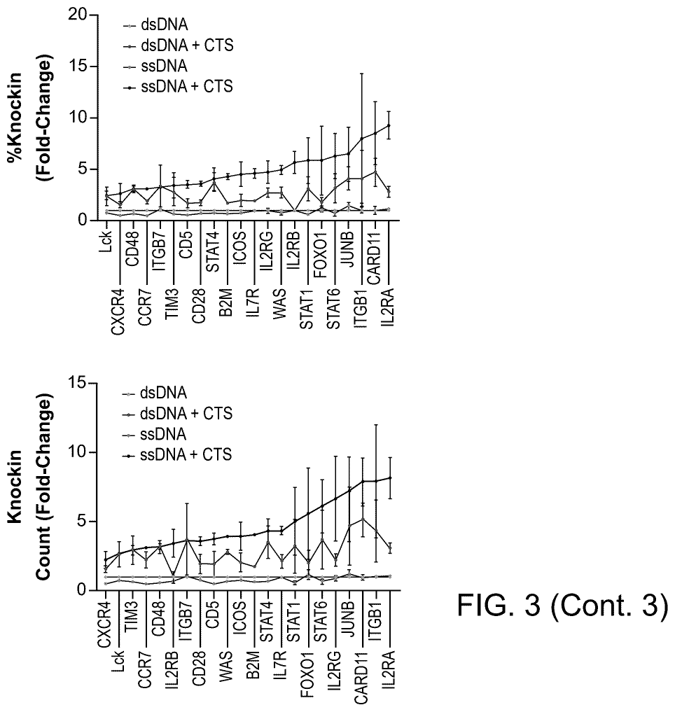

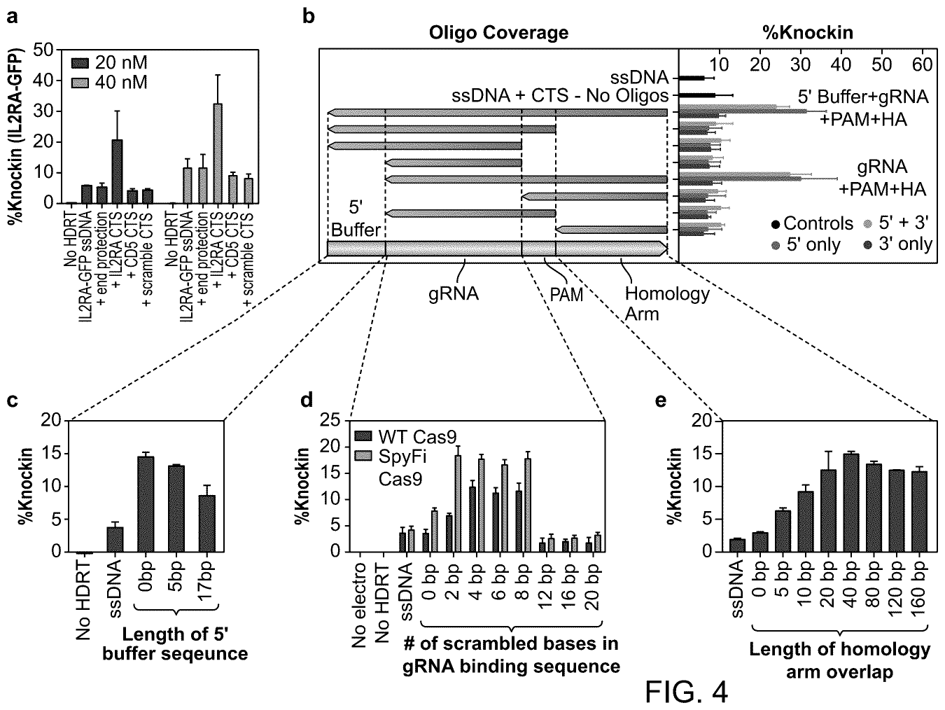

To learn rules regarding the precise sequences required for ssCTS-enhanced HDR, we evaluated variations of two constructs targeting either an IL2RA-GFP fusion to the IL2RA gene (˜2.3 kb, FIG. 1E) or a large version of the CD5-HA knock-in including >1 kb homology arms (˜2.7 kb, FIG. 3A). We first evaluated the specificity of the CTS sequences by replacing them with a mismatched CTS site specific for the alternative RNP, an equivalent length of dsDNA within the homology arm (“end protection”), or a CTS site with scrambled gRNA sequence (FIG. 4A, FIG. 3B). For both constructs, only the matching CTS recognized by the cognate RNP increased knock-in efficiency, suggesting specific recognition of the gRNA sequence.

We next examined closely which components of the CTS required dsDNA by annealing oligos of varied lengths and coverage (FIG. 4B, FIG. 3C). Coverage of the gRNA sequence, PAM, and a stretch of nucleotides within the homology arm downstream of the CTS site were each required for enhancement of knock-in efficiency while coverage of nucleotides upstream of the gRNA sequence in the 5′ buffer region was not. In agreement, inclusion of additional buffer sequence upstream of the CTS was not required at all and may in fact reduce the knock-in efficiency (FIG. 4C, FIG. 3D). Surprisingly, we saw that inclusion of a CTS on the 3′ end of both large ssCTS constructs provided no independent or additive benefit in combination with a 5′ CTS. Similar findings were seen within our short HDRT screen (FIG. 1B-C, “c” versus “d”). These intriguing results suggest only the 5′ CTS is functional in these designs which could reflect requirements for RNP binding and orientation, intracellular trafficking, or interference with repair machinery during 3′ annealing of long ssDNA.

We further examined the requirements for gRNA recognition by generating CTS sites with a variable number of scrambled bases at the 5′ end of the 20 bp gRNA recognition sequence (FIG. 4D, FIG. 3E). We found that for WT Cas9, the enhancement in knock-in efficiency was maximal with inclusion of 4-8 mismatched nucleotides. This level of mismatch likely allows the Cas9 RNP to bind without cleaving the CTS, as has been shown for truncated gRNAs. The pattern was similar with the high fidelity “SpyFi” Cas9 variant produced by Aldevron/IDT, which has been developed to reduce off-target cuts in clinical gene editing applications. Finally, we evaluated the length of the complementary oligonucleotide coverage within the downstream homology arm, demonstrating optimal knock-in when >20-40 bp of the homology arm has complementary sequence in the corresponding oligo (FIG. 4E, FIG. 3F). Taken together, these data establish design rules for end oligos to introduce CTS into large ssDNA templates and boost HDR outcomes, demonstrating that optimal designs need only incorporate a single CTS site on the 5′ end with a short stretch of dsDNA covering the gRNA recognition site, the PAM sequence, and ˜20 bp of the downstream homology arm (FIG. 4B, FIG. 3C). RNPs were formulated with Cas9-NLS proteins and either PGA or ssDNAenh anionic polymers prior to incubation with CTS templates. Full sequences for each component can be found in the table directly below (gRNA sequences) and at the bottom of the example (HDRT and CTS sequences and plasmid sequences).

TABLE A
Misc

| | | | SEQ |
|---|---|---|---|
| gRNA ID | Target | gRNA Target Sequence | ID NO: |
| G147 | CLTA | GAACGGATCCAGCTCAGCCA | 12 |
| G377 | IL2RA | TGAGCAGTCCCCACATCAGC | 13 |
| G520 | CTLA-4 | gatatgacaaacagaagacc | 4 |
| G472 | CD5 | CAGTCGCTTCCTGCCTCGGA | 14 |
| G526 | TRAC | TCAGGGTTCTGGATATCTGT | 15 |
| G527 | TRAC | CTGGATATCTGTGGGACAAG | 16 |

Arrayed Knockin Panel

| | | | SEQ |
|---|---|---|---|
| gRNA ID | Target | gRNA Target Sequence | ID NO: |
| G358: CTLA4-N 1 | CTLA4 | ACACCGCTCCCATAAAGCCA | 76 |
| G360: LAG3-N 1 | LAG3 | GACCATAGGAGAGATGTGGG | 77 |
| G368: CD28-N 1 | CD28 | TCGTCAGGACAAAGATGCTC | 78 |
| G403: CCR2 N 1 | CCR2 | GAGATGTGGACAGCATGTTG | 79 |
| G385: IL7R N 1 | IL7R | tctctcAGAATGACAATTCT | 80 |
| G421: STAT1 N 1 | STAT1 | TTCCCTATAGGATGTCTCAG | 81 |
| G534: JUNB-N2 | JUNB | CGCCCGGATGTGCACTAAAA | 82 |
| G535: IRF4-N1 | IRF4 | CACGCGGGGCATGAACCTGG | 83 |
| G536: FOXO1-N1 | FOXO1 | CACCTGAGGCGCCTCGGCCA | 84 |
| G537: FOXP1-N1 | FOXP1 | GAGTCATGATGCAAGAATCT | 85 |
| G375: IL2RB-N 2 | IL2RB | CAGACGCCAGGACAGAGCAG | 86 |
| G377: IL2RA-N 2 | IL2RA | TGAGCAGTCCCCACATCAGC | 13 |
| G538: ICOS N2 | ICOS | TTTCTGGCAAACATGAAGTC | 87 |
| G364: IL2RG-N 1 | IL2RG | TGGTAATGATGGCTTCAACA | 88 |
| G388: B2M N 2 | B2M | GGCCACGGAGCGAGACATCT | 89 |
| G390: ITGB7 N 1 | ITGB7 | GGGCATGGTGGCTTTGCCAA | 90 |
| G426: STAT6 N 2 | STAT6 | ACCCCACAGAGACATGATCT | 91 |
| G391: CD5 N 1 | CD5 | AGGCCAGAAACCATGCCCAT | 92 |
| G393: CCR7 N 1 | CCR7 | CCAGAGAGCGTCATGGACCT | 93 |
| G395: CD48 N 1 | CD48 | GAAGGAAGCATGTGCTCCAG | 94 |
| G366: PDCD1-N 1 | PDCD1 | TCCAGGCATGCAGATCCCAC | 95 |
| G427: STAT4 N 1 | STAT4 | TCTTTTATAGCATGTCTCAG | 96 |
| G424: STAT2 N 2 | STAT2 | CAGAGCCCAAATGGCGCAGT | 97 |
| G362: TIM3-N 1 | TIM3 | AAAGGGAAGATGTGAAAACA | 98 |
| G406: CXCR4 N 2 | CXCR4 | CTGCAAAAGAGGCAAAGGAA | 99 |
| G407: IL2 N 1 | IL2 | CAACTCCTGCCACAATGTAC | 100 |
| G411: Lck N 1 | LCK | CCCTCAGGGACCATGGGCTG | 101 |
| G416: CARD11 N 2 | CARD11 | GCCGAGTACCTGGCATGGAG | 102 |
| G539: WAS N 3 | WAS | TCCCATTGGGCCCCCACTCA | 103 |
| G420: ITGB1 N 2 | ITGB1 | ATCAGTCCAATCCAGAAAAT | 104 |
| G402: CD27 N 2 | CD27 | CCAGGGATGTGGCCGTGCCA | 105 |

Primary Immunodeficiency Panel

| gRNA ID | Target | gRNA Target Sequence | SEQ ID NO: |
|---|---|---|---|
| CD7 g1 | CD7 | TCAGCTGCACTCGCCCGGCT | 17 |
| CD7 g2 | CD7 | CCAGAAGCAGGGGCAGCAGC | 18 |
| LRBA g1 | LRBA | CCATTGCTAGCTCACGATAA | 19 |
| LRBA g2 | LRBA | CCTTTATCGTGAGCTAGCAA | 20 |
| CD40LG g1 | CD40LG | AAAGTTGAAATGGTATCTTC | 21 |
| CD40LG g2 | CD40LG | ATGCTGTGTTAAAGTTGAAA | 22 |
| MAGT1 g1 | MAGT1 | ACGTAATGTGAGGGGTCTCC | 23 |
| MAGT1 g2 | MAGT1 | GGCCCCTTGCCTTTCCTCAT | 24 |
| WASP g1 | WASP | CTTTCTGCCCTTGTCTTCTC | 25 |
| WASP g2 | WASP | ATGAGTGGGGGCCCAATGGG | 26 |
| RHOH g1 | RHOH | GGACTTCAGAGTAGGACAGC | 27 |
| RHOH g2 | RHOH | TCAGAGTAGGACAGCAGGCT | 28 |
| BCL10 g1 | BCL10 | AGGTCCTCCTCGGTGAGGGA | 29 |
| BCL10 g2 | BCL10 | CTCGGTGAGGGACGGTGCGG | 30 |
| ITGB2 g1 | ITGB2 | AGCATGTCCTGTGGAGGGAA | 31 |
| ITGB2 g2 | ITGB2 | AGGCCCAGCATGTCCTGTGG | 32 |
| IL10RA g1 | IL10RA | CTGGGCGCGCCGCATCGTCC | 33 |
| IL10RA g2 | IL10RA | GCCAGCAGCACTACGAGGCA | 34 |
| SAP g1 | SAP | CCTTGCACAGTTCTCCTCCT | 35 |
| SAP g2 | SAP | CAGCCACTGCGTCCATGGCC | 36 |
| PI3KCD g1 | PI3KCD | TTTTTAGGACAACTGTCATC | 37 |
| PI3KCD g2 | PI3KCD | TAACAACGCAGGATGCCCCC | 38 |
| CD4 g1 | CD4 | TCGGCAAGGCCACAATGAAC | 39 |
| CD4 g2 | CD4 | GCTCAGGCCCCTGCCTCCCT | 40 |
| CD5 g1 | CD5 | AGCAGGTACAAGGTGGCCAG | 41 |
| CD5 g2 | CD5 | CATGGGGTCTCTGCAACCGC | 42 |
| STAT3 g1 | STAT3 | TTGGGACCCCTGATTTTAGC | 43 |
| STAT3 g2 | STAT3 | GCTGCTGTAGCTGATTCCAT | 44 |
| ZAP70 g1 | ZAP70 | TGCTGCCGTAGAAGAAGGGC | 45 |
| ZAP70 g2 | ZAP70 | GAGATGCTGCCGTAGAAGAA | 46 |
| PI3KR1 g1 | PI3KR1 | AACCAGGCTCAACTGTTGCA | 47 |
| PI3KR1 g2 | PI3KR1 | ATTTGCAAACATGAGTGCTG | 48 |
| STIM1 g1 | STIM1 | ACGCATACATCCATGACTCT | 49 |
| STIM1 g2 | STIM1 | CAGTCAACATGCGTCTGATG | 50 |
| CD45 g1 | CD45 | CACAAATACATGGTCATATC | 51 |
| CD45 g2 | CD45 | GTATTTGTGGCTTAAACTCT | 52 |
| CD3D g1 | CD3D | GAACATAGCACGTTTCTCTC | 53 |
| CD3D g2 | CD3D | GGATGAGTTCCGCTGGGAGA | 54 |
| CD2 g1 | CD2 | ACATGGAAAGCTCATCTTAG | 55 |
| CD2 g2 | CD2 | TACATGGAAAGCTCATCTTA | 56 |
| CD3G g1 | CD3G | CCATGTCAGTCTCTGTCCTC | 57 |
| CD3G g2 | CD3G | CCGGAGGACAGAGACTGACA | 58 |
| CD3Z g1 | CD3Z | AGGGAAAGGACAAGATGAAG | 59 |
| CD3Z g2 | CD3Z | GCCTCCCAGCCTCTTTCTGA | 60 |
| CD3E g1 | CD3E | AGATGCAGTCGGGCACTCAC | 61 |
| CD3E g2 | CD3E | CCATGAAACAAAGATGCAGT | 62 |

### Example 4. SsCTS Templates Provide a Flexible and Powerful Approach to Enhance HDR in Primary Human Cells

Using optimized ssCTS designs, we next assessed performance across a broad array of genomic loci, knock-in constructs, and primary hematopoietic cell types. We evaluated CTS templates in a variety of clinically relevant primary cell types using an mCherry fusion construct targeting a gene not expected to affect cell fitness (Clathrin, CLTA). Knockin at this locus demonstrated no selective growth advantage in primary human T. ssCTS templates significantly increased knock-in efficiency, live cell counts, and absolute yield of knock-in cells across all primary human cell types evaluated here including CD4+ T cells, CD8+ T cells, regulatory T cells (Treg), NK cells, B cells, CD34+ hematopoietic stem cells (HSC) and gamma-delta T cells (γδ).

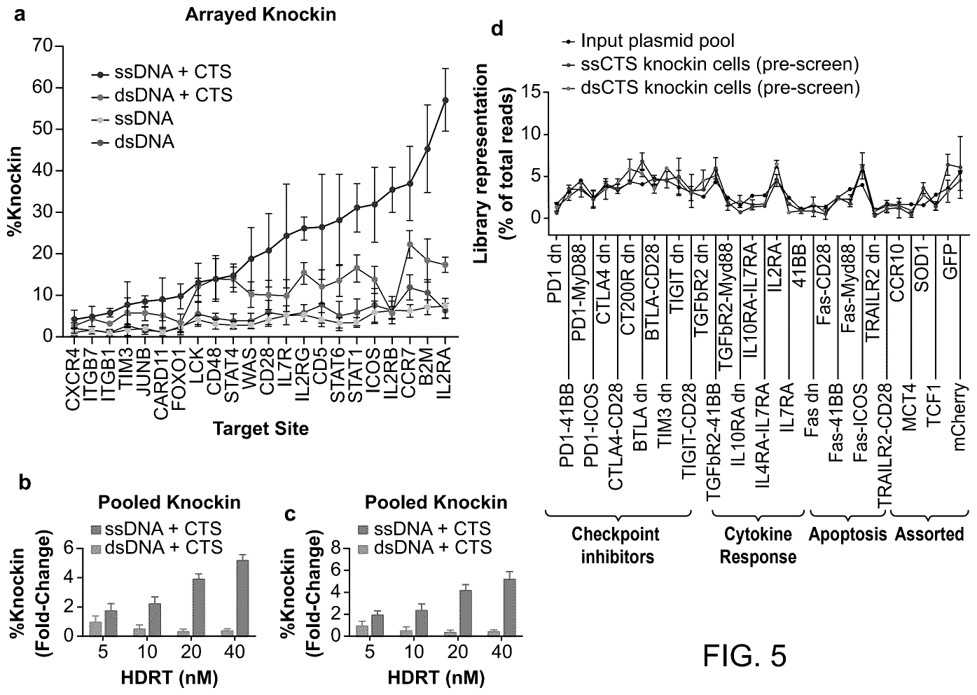

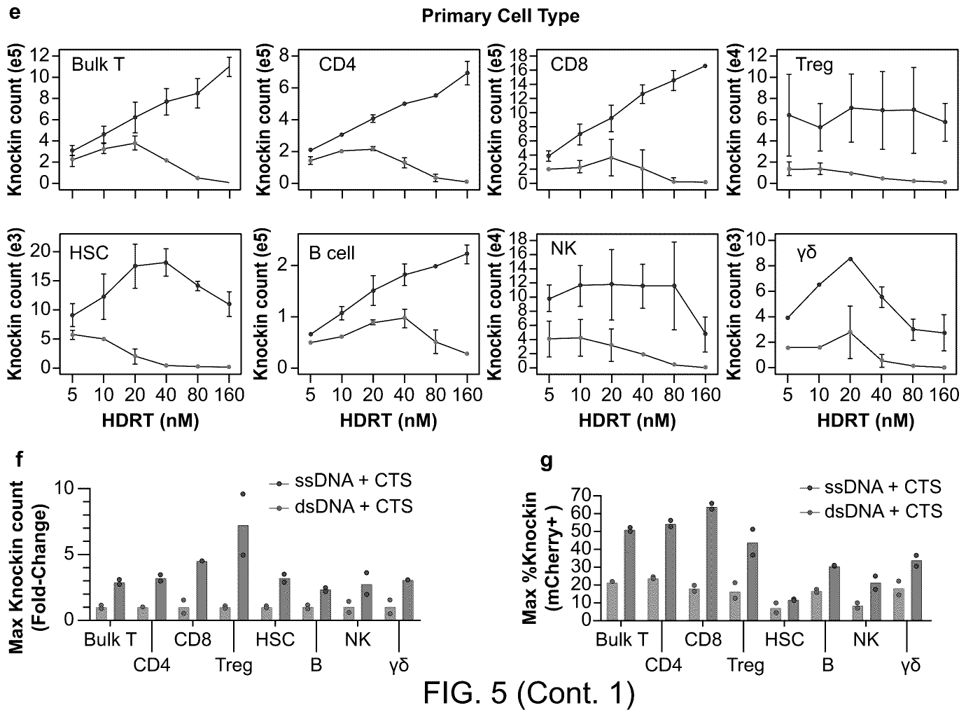

We evaluated an arrayed panel of knock-in constructs in primary human T cells targeting a detectable tNGFR fusion at the 5′ end of 22 different genes (FIG. 5A). The majority of ssCTS constructs outperformed alternative HDRT variations for both knock-in efficiency (up to 5-fold increase) and absolute knock-in counts (up to 3-fold increase) with only a few exceptions that appeared equivalent to dsCTS constructs (FIG. 5A, FIG. 3G). We next evaluated performance with a pooled library of knock-in constructs targeting an NY-ESO-1 specific TCR and additional gene products to the endogenous TRAC locus, as previously reported by our group for use in functional knock-in screens (FIG. 5B-D). Knock-in pools provide a powerful approach for high-throughput screening and allowed us to assess performance with a diverse population of large knock-in templates ranging from 2.6-3.6 kb. Knock-in efficiency and absolute knock-in counts were both increased by >5-fold in comparison to optimal dsCTS concentrations, significantly increasing coverage for each individual construct while retaining consistent representation of the initial library in the final knock-in population (FIG. 5B-D). Finally, we evaluated performance across a variety of clinically relevant primary cell types including CD4+ T cells, CD8+ T cells, regulatory T cells (Treg), NK cells, B cells, hematopoietic stem cells (HSC) and gamma-delta T cells using an mCherry knock-in construct targeting the clathrin light chain A (CLTA) gene (FIG. 5E-G). In all evaluated cell types ssCTS templates demonstrated significantly lower toxicity, increased knock-in efficiency, and generated higher absolute knock-in cell yields.

### Example 5. Evaluation of Small Molecule Inhibitors in Combination with ssCTS Templates for High-Efficiency T Cell Engineering at Primary Immunodeficiency (PID) Disease Loci

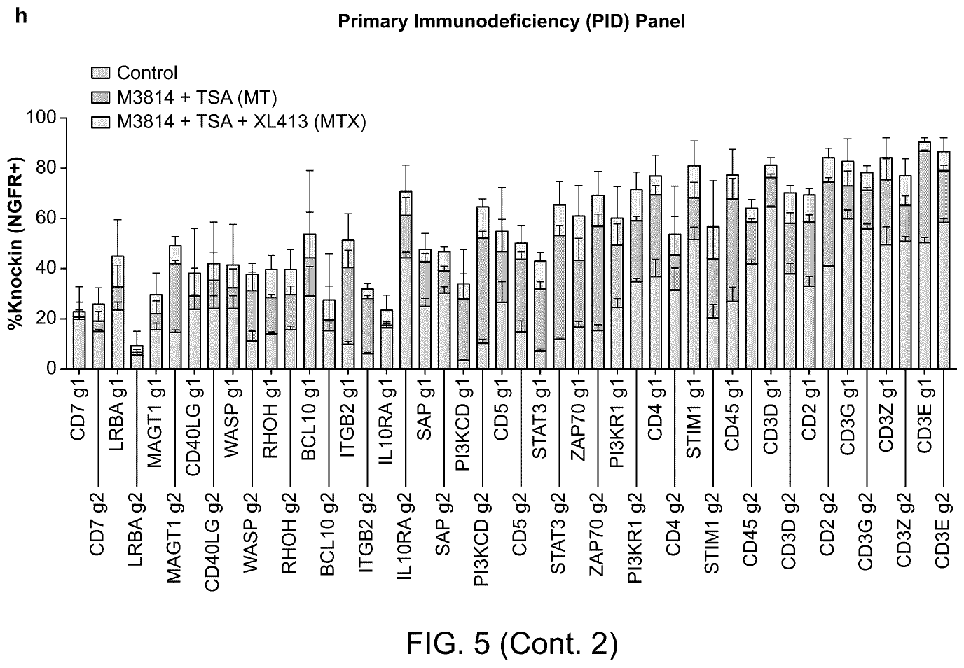

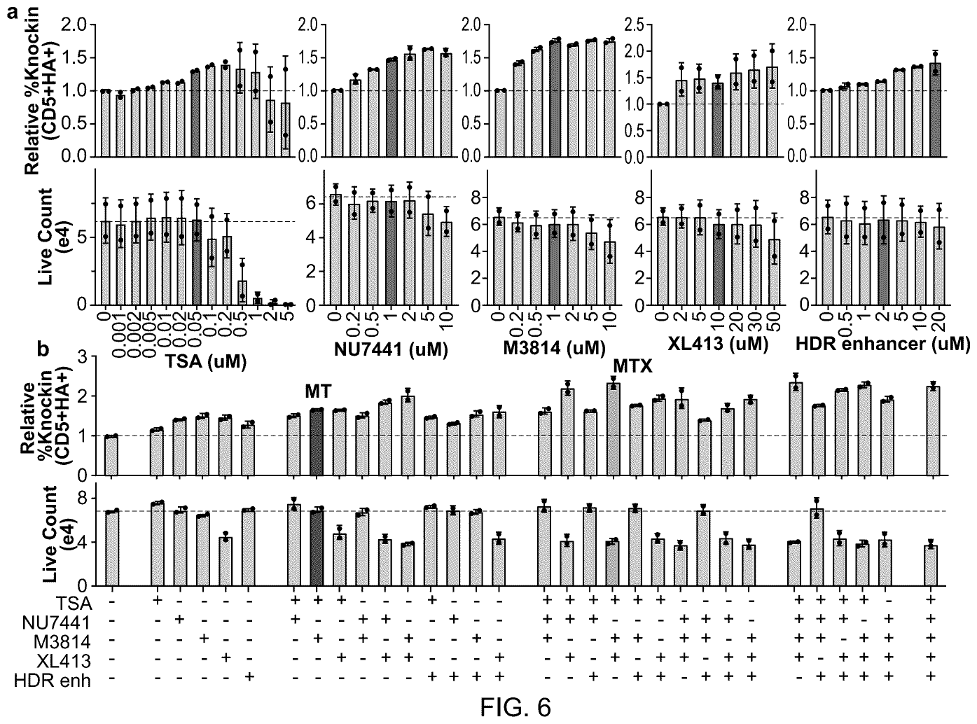

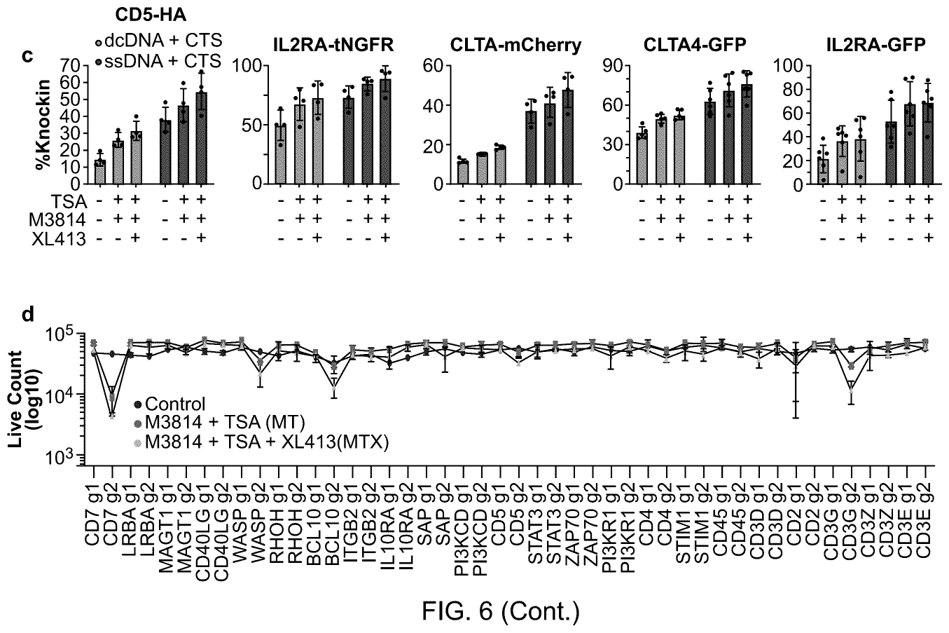

We next evaluated a panel of small molecule inhibitors reported to enhance knock-in efficiency in primary human T cells including the DNA-PK inhibitors NU7441 and M3814, the HDAC class I/II Inhibitor Trichostatin A (TSA), the CDC7 inhibitor XL413, and IDT's proprietary Alt-R HDR Enhancer which is described as a NHEJ inhibitor. Using our short ssDNA CD5-HA knock-in construct (FIG. 2A-B), each was titrated in isolation and then evaluated in combination to identify effects on knock-in efficiency and live cell counts (FIG. 6A-B). At optimal concentrations, M3814 showed the largest effect size (˜49% increase), followed by XL413 (˜46% increase), NU7441 (˜43% increase), IDT's HDR Enhancer (˜29% increase), and TSA (˜16% increase). Live cell counts were generally unaffected at the chosen concentrations except for combinations involving XL413, which demonstrated an ˜50% reduction in cell counts at day 4 post-electroporation that may reflect XL413's mechanism as a transient cell cycle inhibitor rather than overt cytotoxicity (FIG. 6B). NHEJ inhibitor combinations (M3814, NU7441, IDT HDR Enhancer) did not demonstrate further improvements above the highest individual component, consistent with overlapping mechanisms of action. In contrast, addition of TSA or XL413 did demonstrate additional improvements in combination with NHEJ inhibitors. The M3814/TSA (MT) combination provided the largest increase in knock-in efficiency without affecting live cell counts (˜65% increase) and the M3814/TSA/XL413 (MTX) combination demonstrated the highest absolute increase in knock-in efficiency (˜134% increase) albeit with XL413-mediated reduction in total cell counts. Finally, we evaluated whether the benefits of ssCTS templates and small molecule inhibitors could be combined using a variety of constructs ranging from 1.5-2.7 kb (FIG. 6C). Encouragingly, each construct demonstrated increased knock-in efficiency with ssCTS templates that was further enhanced by the inclusion of MT and MTX inhibitor combinations, in some cases generating knock-in efficiencies >90%.

We further examined repair outcomes at the genetic level by amplicon sequencing of the CD5 target locus with different versions of the small CD5-HA templates and inhibitor cocktails. Sequencing and flow-based quantifications both demonstrated stepwise increases in knock-in rates with each inhibitor that was additive to the increases seen with CTS sequences in both ssDNA and dsDNA templates. The ratio of perfect:imperfect HDR events was also similar across the different types of templates. Intriguingly, treatment combinations except for those involving XL413. All that included the M3814 DNA-PK inhibitor were associated with decreased frequency of indels (especially small indels characteristic of NHEJ), along with reduced frequency of imperfect HDR events. Inclusion of HDR templates was associated with preferential reduction in the larger deletions characteristic of MMEJ (while smaller indels characteristic of NHEJ were more refractory in the absence of DNA-PK inhibition), in agreement with recent reports.

We next asked whether small-molecule inhibitors could be combined with large ssCTS templates (ranging from 1.5-2.7 kb) to enhance knock-in engineering. Encouragingly, each ssCTS template demonstrated increased knock-in efficiencies that were enhanced further by the inclusion of MT and MTX inhibitor combinations with XL413 demonstrated an ˜50% reduction in cell counts at Day 4 and an ˜20% reduction in cell counts at Day 11. This reduction in cell count may be related to XL413's mechanism as a transient cell cycle inhibitor rather than overt cytotoxicity, in some cases generating knock-in efficiencies >90%.

Encouraged by these results, we sought to evaluate these approaches more broadly and at clinically relevant target sites that could lead toward diagnostic or therapeutic advances. We developed a panel of knock-in constructs targeting genes associated with monogenic disease-causing mutations affecting T cell function or relevant controls. These diseases are part of a spectrum of increasingly recognized genetic disorders, referred to as Primary Immunodeficiencies (PID) or Inborn Errors of Immunity (IEI), that disrupt the healthy immune system, presenting with severe infections, autoimmune disease, and malignancy. Within this panel, we examined 44 different tNGFR constructs targeting 22 genes (2 gRNA targets per gene) using ssCTS templates +/−MT and MTX inhibitor combinations (FIG. 5H). This analysis demonstrated nearly universal increases in knock-in efficiency with MT that were further enhanced with the MTX combination, achieving knock-in rates >50% for these large constructs at 15/22 genes examined and >80% at 6/22 genes. The effect size of inhibitors varied among target loci, with some sites demonstrating relatively little increase (e.g. CD7 g1) and others showing up to 7-fold increases (e.g. PI3KCD g2). Live cell counts were comparable at day 5 post-electroporation with a few notable exceptions demonstrating significant toxicity with both combinations (e.g. CD7 g2, WASP g2, CD3G g2) (FIG. 6D). Altogether these findings support broad application of ssCTS templates and inhibitor combinations at relevant disease loci, in some cases demonstrating nearly pure populations of knock-in cells (>80-90%) (FIG. 5H, FIG. 6C). This sets the stage for diagnostic and therapeutic applications of non-viral human T cell engineering that require a high purity or yield of knock-in cells at specific disease loci.

### Example 6. Universal Gene Replacement Strategies for Therapeutic and Diagnostic Human T Cell Editing

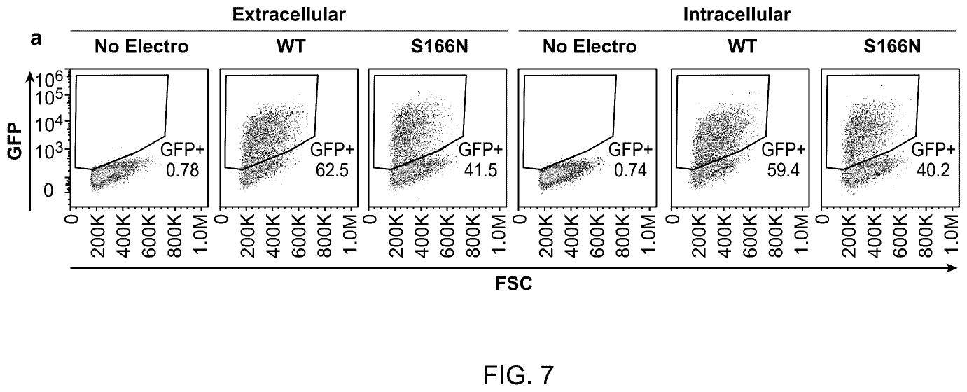

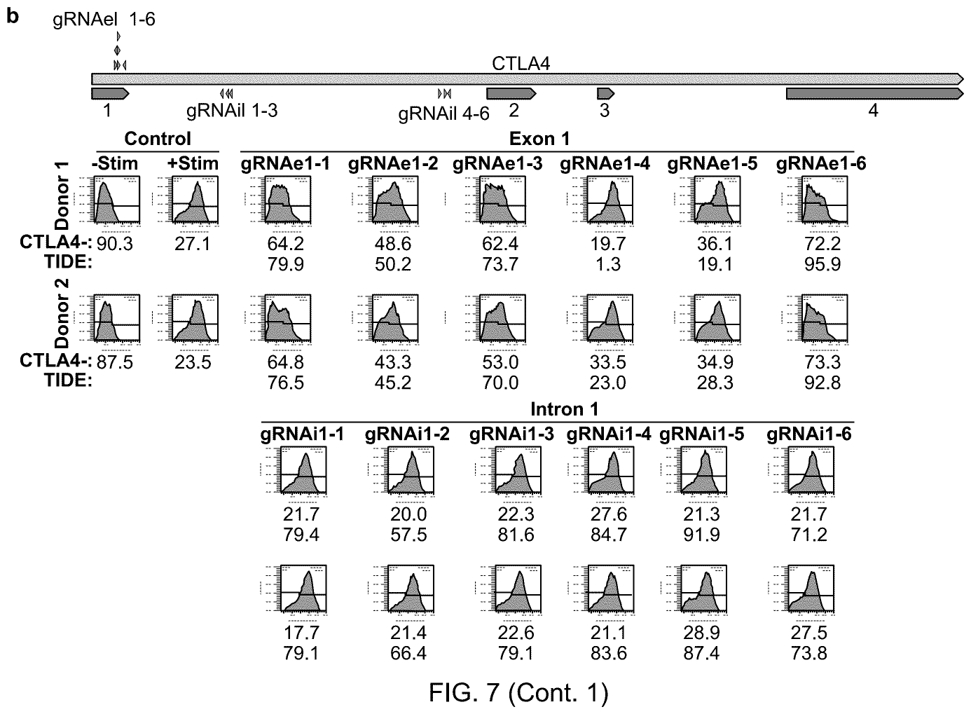

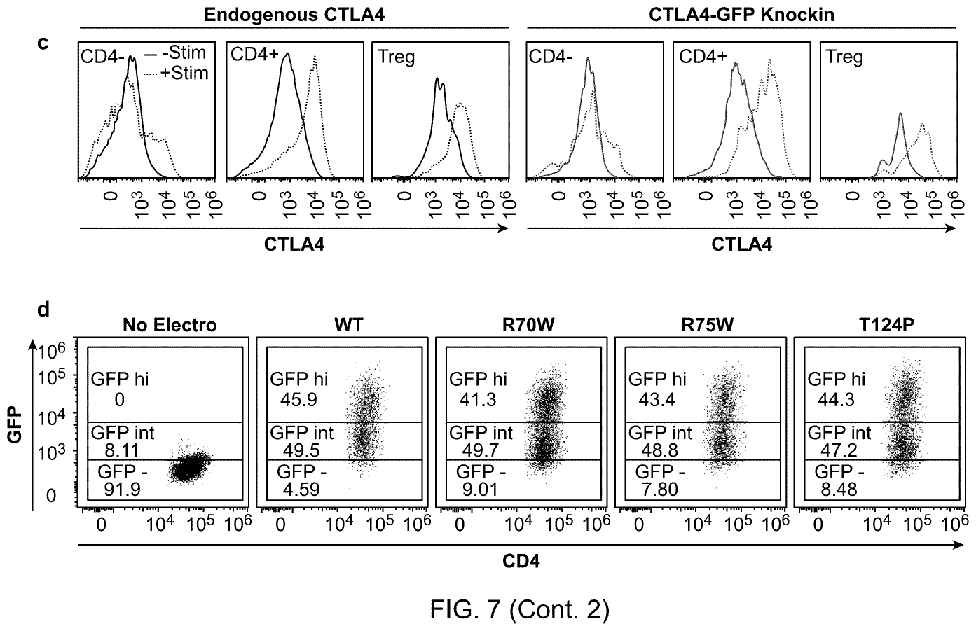

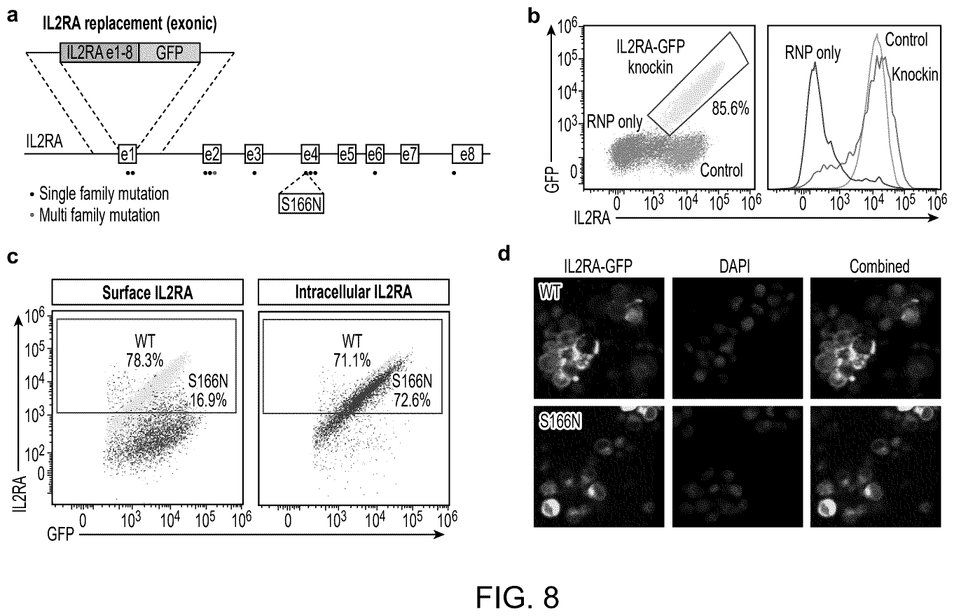

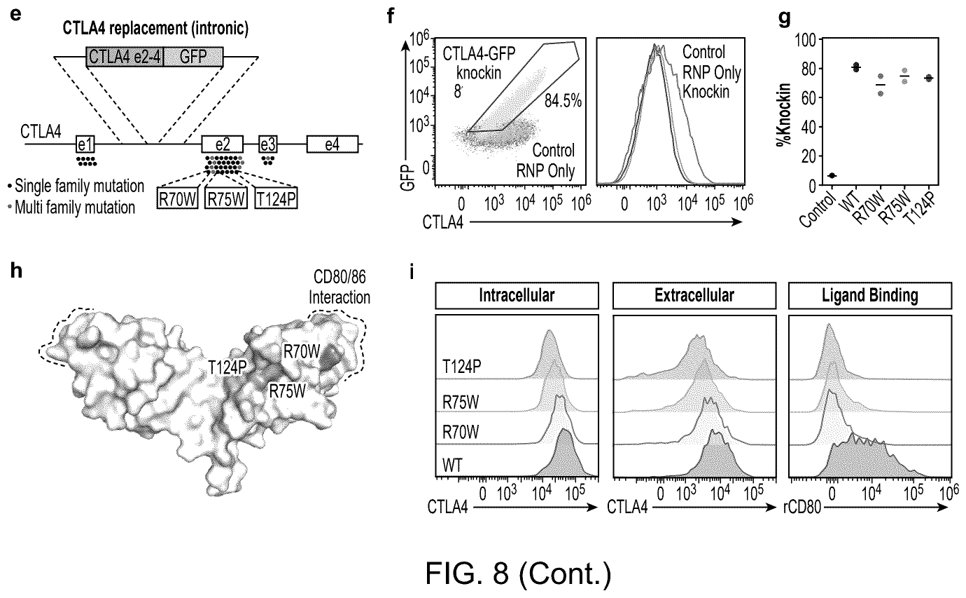

To explore potential clinical applications with large non-viral templates, we chose to examine whole open reading frame (ORF) insertions for two genes, IL2RA and CTLA4, that have been identified in families with monogenic immune disorders characterized by severe multi-organ autoimmunity. Although disease-causing mutations are widely distributed throughout these genes, many of these families could potentially be treated by a universal ORF replacement strategy (FIG. 8A, 8E). For each construct, we included a GFP fusion at the 3′ end to facilitate detection of the knock-in protein. We have previously reported targeted gene corrections for a family with loss-of-function mutations in exon 4 and exon 8 of the IL2RA gene. While we achieved knock-in efficiencies >30% with this approach, each site required a custom gRNA and HDRT which prevents extension to families with alternative IL2RA mutations. In contrast, a whole ORF knock-in at exon 1 of the IL2RA gene could potentially ameliorate any of the 11 previously reported mutations causing IL2RA deficiency (FIG. 8A). Using a ssCTS template and the MTX inhibitor combination, we achieved >80% knock-in of a ˜2.3 kb whole ORF IL2RA-GFP fusion construct (FIG. 8B). The knock-in protein demonstrated nearly indistinguishable expression levels compared to endogenous protein. This whole ORF knock-in approach could allow for rapid functional testing and characterization of patient mutations or variants of unknown significance (VUS). To demonstrate this diagnostic potential, we modified the knock-in construct to encode a previously described disease-causing mutation in exon 4 of IL2RA, c.497 G>A (S166N), which was reported to eliminate surface expression while retaining cytoplasmic protein. In agreement with what has been reported in patient cells, we found that the GFP+S166N population demonstrates a near complete absence of surface IL2RA with readily detectable intracellular IL2RA comparable to WT levels (FIG. 8C, FIG. 7A). Fluorescence microscopy revealed that S166N protein formed distinct perinuclear aggregates consistent with intracellular retention and contrasting with the diffuse cytoplasmic and surface T1L2RA seen with WT knock-ins (FIG. 81D). These results highlight the diagnostic and therapeutic potential of targeted ORF insertion within the endogenous gene, an approach which may be extended to include a number of alternative targets or additional noncoding elements.

As a further example, we examined an ORF insertion within the CTLA4 gene (FIG. 8E). CTLA4 deficiency is caused most frequently by a haploinsufficiency with a disease-causing mutation on only 1 of 2 alleles. Exon-targeting strategies generate indels which could disrupt the normal allele and worsen disease. To avoid this possibility, we screened a panel of gRNA in intron 1, set forth in Table 3, to identify targets which cut efficiently without disrupting protein expression (FIG. 7B).

TABLE 3

| | | SEQ | %CTLA4 protein | |
|---|---|---|---|---|
| | | ID | loss above | |
| | gRNA target sequence | NO: | background | %indel |
| G509: | CCTTGTGCCGCTGAAATCCA | 63 | 39.2 | 78.2 |
| CTLA4e1-1 | | | | |
| G510: | CCTTGGATTTCAGCGGCACA | 64 | 16.65 | 47.7 |
| CTLA4e1-2 | | | | |
| G511: | GCACAAGGCTCAGCTGAACC | 65 | 32.4 | 71.85 |
| CTLA4e1-3 | | | | |
| G512: | CTCAGCTGAACCTGGCTACC | 66 | 1.3 | 12.15 |
| CTLA4e1-4 | | | | |
| G513: | TGAACCTGGCTACCAGGACC | 67 | 10.2 | 23.7 |
| CTLA4e1-5 | | | | |
| G514: | AGAAAAAACAGGAGAGTGCA | 68 | 47.45 | 94.35 |
| CTLA4e1-6 | | | | |
| G515: | TTGTATGCCAAACAGACTGG | 69 | 0 | 79.25 |
| CTLA4i1-1 | | | | |
| G516: | TGCATCTGAAACCCACACGT | 70 | 0 | 61.95 |
| CTLA4i1-2 | | | | |
| G517: | CTCTGAAGAGCTGAAAACTG | 71 | 0 | 80.35 |
| CTLA4i1-3 | | | | |
| G518: | GAAAAAAATGAAGGACATGG | 72 | 0 | 84.15 |
| CTLA4i1-4 | | | | |
| G519: | ACATATTTCATGGTAGAGCC | 73 | 0 | 89.65 |
| CTLA4i1-5 | | | | |
| G520: | GATATGACAAACAGAAGACC | 4 | 0 | 72.5 |
| CTLA4i1-6 | | | | |

The chosen gRNA had no detectable disruption of endogenous CTLA4 protein and the associated ORF knock-in construct generated knock-in efficiencies of 70-80% with ssCTS templates and MTX inhibitor combination (FIG. 9F-G). This intron-targeting strategy could be used to introduce or correct the majority of reported disease-causing mutations in CTLA4 excluding those upstream of the target site (FIG. 9E). Variations in protein expression by cell type and in response to stimulation matched the endogenous protein, although basal knock-in protein levels were slightly higher which may reflect differences between the SV40 3′UTR used in this construct and the endogenous 3′UTR (FIG. 7C). To evaluate diagnostic capabilities with known CTLA4 mutations, we generated knock-in constructs with 3 previously reported disease-causing mutations: R70W, R75W, and T124P. Cells were gated for the highest levels of GFP expression to enrich for homozygous knock-ins and then evaluated for surface protein, intracellular protein, and ligand binding using recombinant CD80 in activated CD4+ T cells (FIG. 7D, FIG. 8G-I). All three mutations significantly reduced ligand binding despite variable levels of surface expression, in agreement with prior reports demonstrating reduced ligand uptake in heterozygous patient cells or engineered cell lines. Altogether, these approaches provide a powerful method for evaluating patient mutations at endogenous loci with the potential for adaptation to high-throughput screening and high efficiency therapeutic gene replacement strategies.

### Example 7. Development of a GMP-Compatible Manufacturing Process for Non-Viral Genome Engineered T Cell Therapies

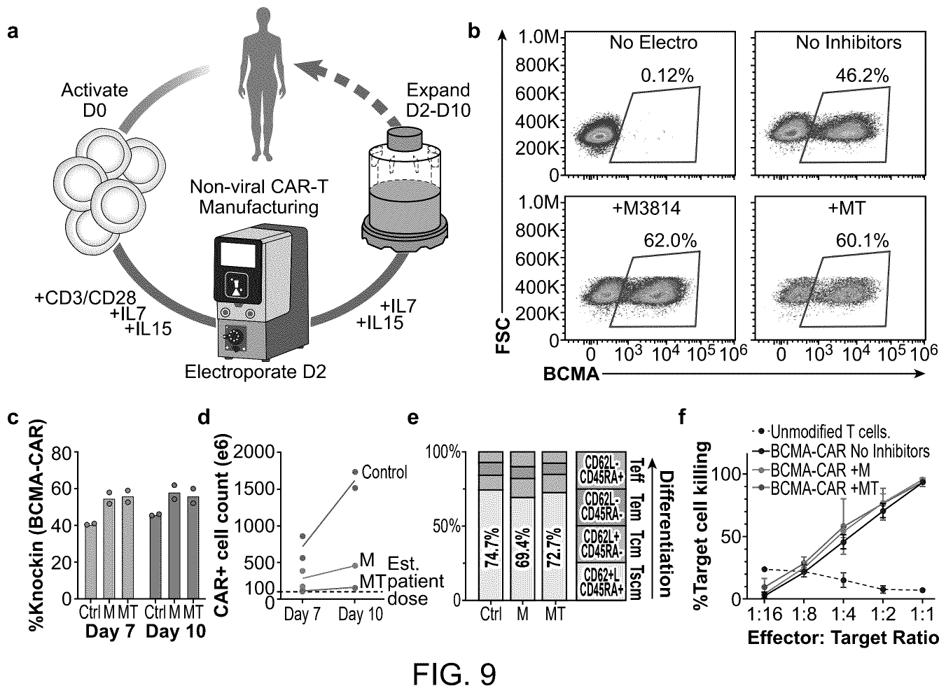

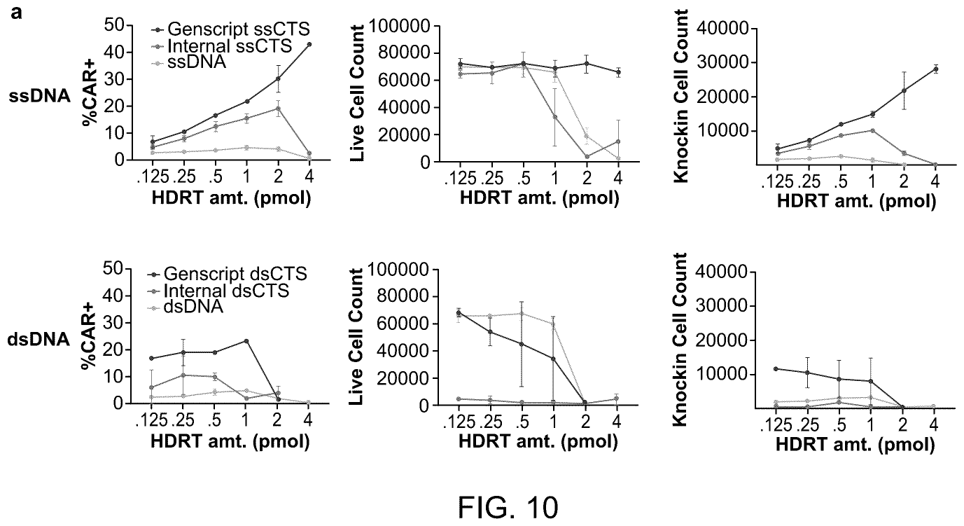

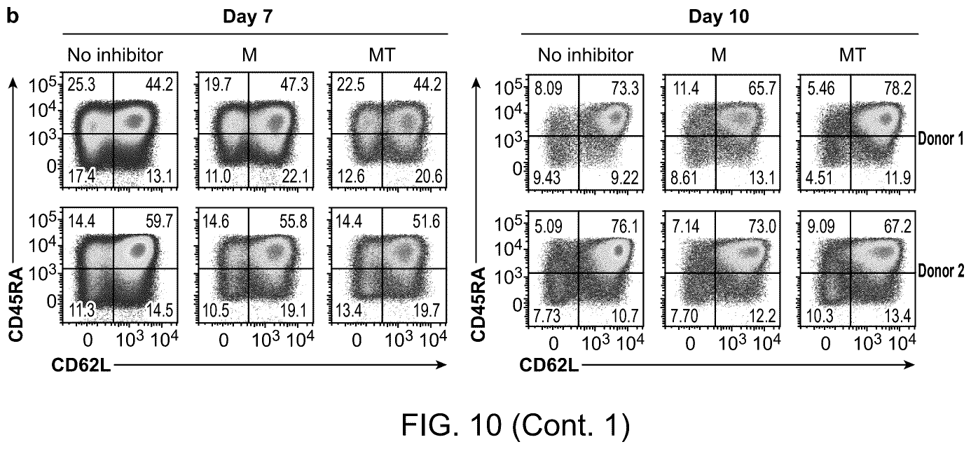

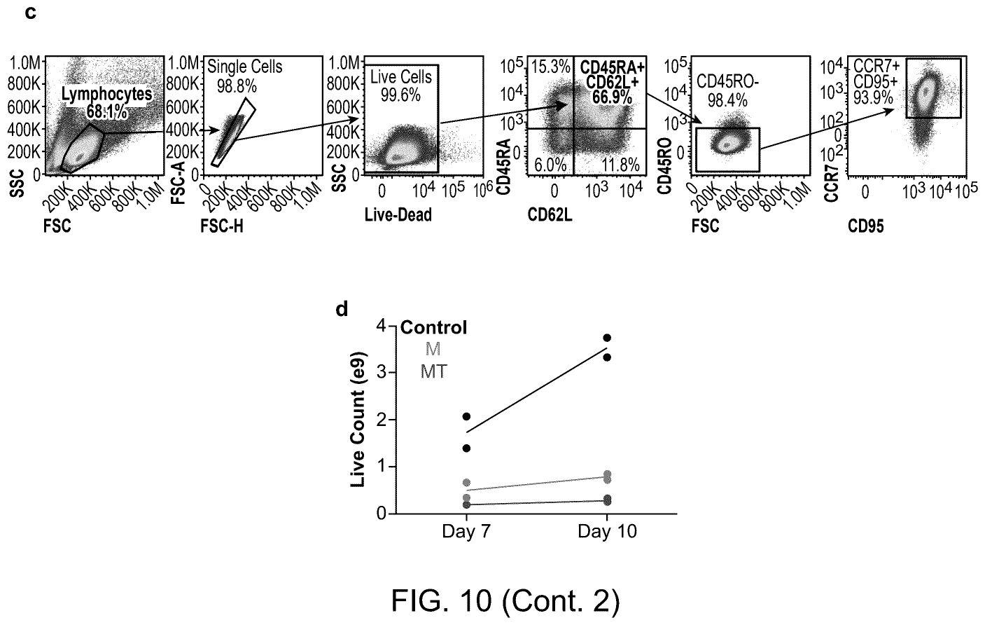

Finally, we sought to generate a clinical-grade process for fully non-viral knock-in of large therapeutic constructs. One of the most immediate applications with demonstrated functional benefit is targeting a CAR insertion to the endogenous TRAC locus. This approach greatly enhanced the potency of CD19-specific CAR-T cells in preclinical studies and reduced T cell exhaustion through tightly regulated expression driven by the gene regulatory elements governing normal TCR expression. In contrast to the original rAAV-mediated methods, we adapted this strategy to make use of ssCTS templates targeting the BCMA antigen, a promising target for treatment of Multiple Myeloma that has recently seen FDA-approval for viral CAR-T products (FIG. 1F). Clinical translation requires transitioning to Good Manufacturing Practice (GMP) compliant reagents, equipment, and processes. For electroporations, we used the Maxcyte GTx platform which provides a GMP-compatible electroporation device with access to FDA Master File along with sterile single-use cuvettes and assemblies that are scalable to the large numbers of cells needed for manufacturing a full patient dose. For genome editing reagents, we used research-grade equivalents that are each available at GMP-grade, including SpyFi Cas9 (a high fidelity Cas9 variant produced at GMP-grade by Aldevron) and chemically synthesized sgRNA also produced at GMP-grade by Synthego. We partnered with Genscript to develop a GMP-compatible process for ssCTS template generation. Encouragingly, Genscript templates encoding a BCMA-CAR knock-in were able to be manufactured at large scale and consistently outperformed our internally generated HDRTs, showing lower levels of toxicity and higher knock-in efficiencies for both ssCTS and dsCTS constructs (FIG. 10A).

To demonstrate a large-scale non-viral CAR T manufacturing process, ˜100×10⁶ primary human T cells were isolated from two healthy donors, activated on Day 0 with CD3/CD28 Dynabeads along with IL-7 and IL-15, electroporated on Day 2 using Maxcyte R-1000 cuvettes, then expanded in G-Rex 100M gas permeable culture vessels to Day 7 or Day 10 (FIG. 9A). Average knock-in efficiencies were 40.4% on Day 7 and 45.8% on Day 10. The final yield of CAR+ cells was >5×10⁸ by Day 7 and >1.5×10⁹ by Day 10 for both donors, well within the range needed to generate a full patient dose of ˜100×10⁶ CAR+ cells (FIG. 9B-D). While the addition of small molecule inhibitors improved knock-in efficiencies to >60%, we observed a reduction in live cell counts such that the final yield of CAR+ cells were decreased in comparison to ssCTS templates alone (FIG. 9 B-D, FIG. 10C-D). The majority of CAR+ cells demonstrated an immunophenotype consistent with a T stem cell memory (Tscm) population on day 10 of expansion based on CD45RA/CD62L expression and confirmed with additional markers as CD45RA⁺CD62L⁺CD45RO⁻CCR7⁺CD95⁺ (FIG. 9E, FIG. 10B-C). In vitro assays demonstrated efficient killing of BCMA+MM1S myeloma cell lines in contrast to unmodified T cells expanded from the same donors (FIG. 9F). Altogether, these results demonstrate a fully non-viral manufacturing process capable of high efficiency T cell engineering at clinical scale which may be transitioned to full-GMP manufacturing and quickly adapted toward additional targets.

The ability of CRISPR genome engineering to introduce targeted sequence replacements or insertions in primary human cells holds immense promise for studying disease variants, correction of genetic diseases, and reprogramming cellular functions for the next-generation of cell-based therapeutics. Here we report advances that improve HDR efficiency and yield with large non-viral ssCTS templates and small molecule inhibitor combinations. We apply this technology across diverse genetic loci, knock-in constructs, and primary hematopoietic cell types, demonstrating their utility for the generation of universal gene correction strategies, disease variant modeling, and GMP-compatible manufacturing processes.

ssCTS hybrid repair templates—alone or in combination with small molecule inhibitors—provide a broadly useful tool to promote CRISPR-based HDR. The technology reported here demonstrated >7-fold increases at some sites. However, by testing knock-in across a broad array of target sites, we did observe variation even with different RNPs targeting the same gene. Variable knock-in rates and toxicity could be affected by unique features of the target site (or off-target effects) at the local sequence or epigenetic level. Recent work has highlighted that some gRNA targets exhibit distinct repair pathway preferences. A detailed analysis of repair outcomes at the sequence level, reliance on alternative repair pathways, and evaluation of off-target effects may help identify the source of this variability and inform future design of genome targeting strategies.

The relatively high purity and high yield of live cells achieved here with large genome replacements provides a powerful tool to probe DNA sequence function in primary human cells. We can now directly test the function of individual coding or non-coding genome sequences for mechanistic studies or to confirm the clinical relevance of disease variants. The most recent classification of Inborn Errors of Immunity (aka Primary Immunodeficiencies or PID) from the 2019 International Union of Immunological Societies (IUIS) update identifies >400 monogenic immune disorders with 65 new genes implicated since 2017. Families with these diseases demonstrate a spectrum of mutations scattered throughout these genes and interpretation of novel variants of unknown significance (VUS) is a persistent challenge to diagnosis and appropriate patient management. Routine interrogation of these VUS at endogenous loci within the relevant primary human cell type may now be feasible. Here we demonstrate application of our non-viral approaches at a variety of PID-associated genes, in some cases achieving knock-in efficiencies >80% without selection and allowing us to evaluate the functional consequences of disease-causing mutations within primary human T cells. We further demonstrate the ability to extend these approaches to alternative hematopoietic cell types and large knock-in pools, providing a foundation for high-throughput functional screens that may be used to examine the immense variety of PID-associated genetic variants.

Enhanced CRISPR-based genome targeting with ssCTS templates also provides opportunities to re-write sequences in primary somatic cells to treat patients. Here we show the potential of non-viral approaches to generate high-efficiency universal open reading frame (ORF) replacements for two genes, IL2RA and CTLA4, both associated with severe autoimmunity and immune dysfunction affecting primary human T cells. Flexible, non-viral gene replacement strategies—in hematopoietic stem and progenitor cells or in more terminally differentiated cell types such as T cells—could give more patients access to curative cellular therapies. More broadly, the ability to efficiently knock-in large sequences into specified genome locations opens the door to synthetic reprogramming to generate powerful cellular medicines. Here we demonstrate clinical-scale, non-viral manufacturing of T cells engineered to have chimeric antigen receptors (CARS) expressed under the gene regulatory control of endogenous TCR-alpha, which has been reported to have favorable properties. Eventually, this process should support robust manufacturing of even more complex synthetic gene programs integrated into targeted genome sites to drive potent cell therapy functions for diverse, complex human diseases.

Altogether, we have developed a variety of tools and applications that markedly improve non-viral genome editing and demonstrate the power of these methods to correct, modify, and reprogram primary human cells. We have applied these approaches predominantly toward genome targets relevant for human T cell editing, demonstrating applications for functional genetic screens or therapeutic genome engineering. However, we also show the feasibility of applying ssCTS templates to a range of relevant human cell types and these approaches may be extended for many alternative applications, including targeting the >400 genes associated with a PID or incorporation of a wide variety of novel synthetic biology constructs. These studies demonstrate the capacity of fully non-viral HDR to mediate complex and targeted genome modifications with high efficiency and yield, which is advantageous for a number of research, diagnostic, and manufacturing applications.

### Example 8. Chemical Control of DNA Repair Pathways

Many genome editing approaches make use of nucleases such as Cas9 to generate targeted genomic double-stranded DNA (dsDNA) breaks. These breaks, in turn, drive a variety of DNA repair pathways including non-homologous end joining (NHEJ), microhomology-mediated end joining (MMEJ), or homology directed repair (HDR), which may be harnessed to generate the desired genetic outcome. For multiplexed genome editing, it is frequently desirable to achieve high rates of HDR to generate a “knock-in” at one locus, while simultaneously generating high rates of “knock-out” at alternative genetic loci in order to perform functional screens or improve the safety or performance of cellular therapies.

A knock-in is generated by inclusion of high concentrations of homology directed repair templates (HDRTs) with the intended genetic insert flanked by homology arms matching the target site. Knock-outs occur when no HDRT is available by either NHEJ or MMEJ repair pathways. NHEJ leads to small insertions or deletions (indels) surrounding the cut site, while MMEJ leads to larger deletions driven by microhomologies surrounding the genomic break which remove the intervening sequence during repair. Because NHEJ indels are small and many remain in-frame, they are less likely to disrupt the final gene product. MMEJ is thus a preferable outcome for disrupting a gene and HDR is preferred for generating a knock-in.

These three pathways compete with each other to repair a genomic break, and, here, we demonstrate that using small molecule inhibitors (M3814, NU7441, TSA, and XL413), in isolation or in combination, can drive repair away from less desirable NHEJ outcomes and toward either MMEJ or HDR depending on whether an HDRT is present for each individual target site. With multiplexed editing, this leads to synergistic increases in both knockout and knock-in. The magnitude of both these effects are enhanced by inclusion of each additional inhibitor.

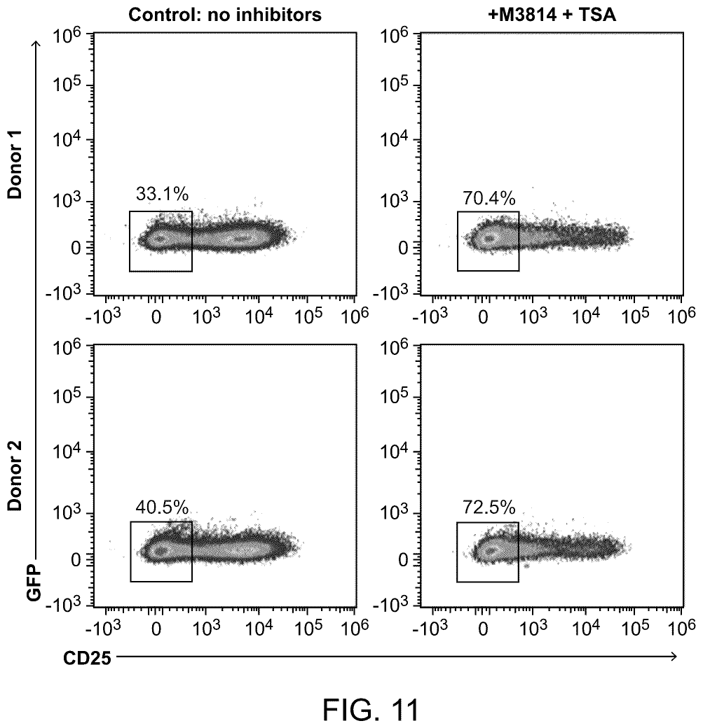

FIG. 11 shows the results of T cells from two independent, healthy donors that were electroporated with RNPs targeting exon 1 of IL2RA. The left column represents cells that were grown in medium and IL-2, while the right column shows samples that were grown in medium, IL-2, M3814, and TSA. Twenty-four hours after electroporations, media from all samples was replaced with fresh growth media and IL-2. Six days after electroporations, cells were collected, stained with fluorescent antibodies, and analyzed by flow cytometry. The samples treated with M3814 and TSA show increased protein knockout of surface CD25 expression.

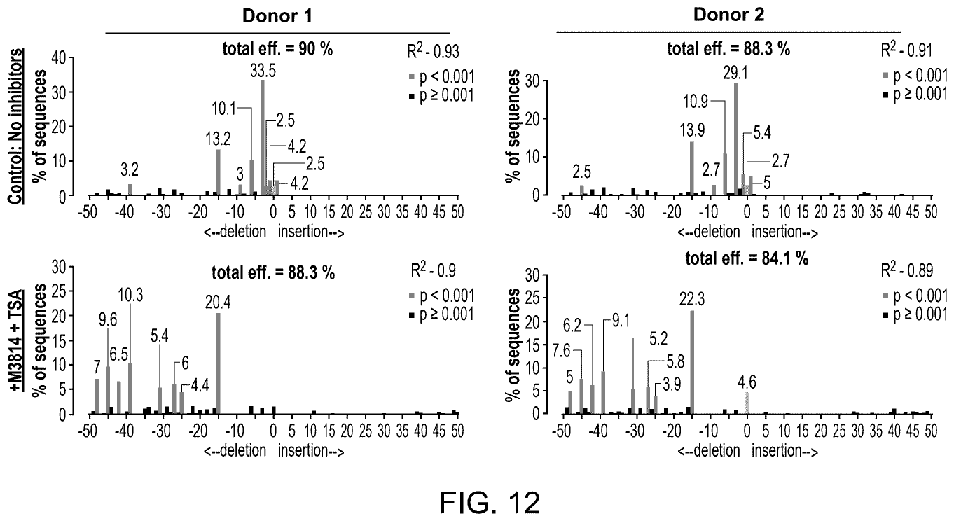

FIG. 12 shows results of T cells from two independent, healthy donors that were electroporated with RNPs targeting exon 1 of IL2RA and immediately treated with either growth media and IL-2 or growth media, IL-2, M3814, and TSA as indicated. Media was refreshed after 24 hours in all samples, and cells were resuspended in growth media and IL-2. Six days after electroporations, genomic DNA was isolated from samples and amplicons were generated using PCR. Amplicons were sequenced and analyzed using TIDE (Tracking of Indels by Decomposition) to determine distinct changes in indel spectrums. The addition of inhibitors abolished almost all small indel outcomes that are characteristic of NHEJ repair pathways and selectively enabled and enriched for much larger deletions that are characteristic of MMEJ repairs. Additionally, the cutting efficiency of G377 is not impacted by the addition of inhibitors, and thus, cannot be attributed to the increased protein knockout observed in FIG. 1.

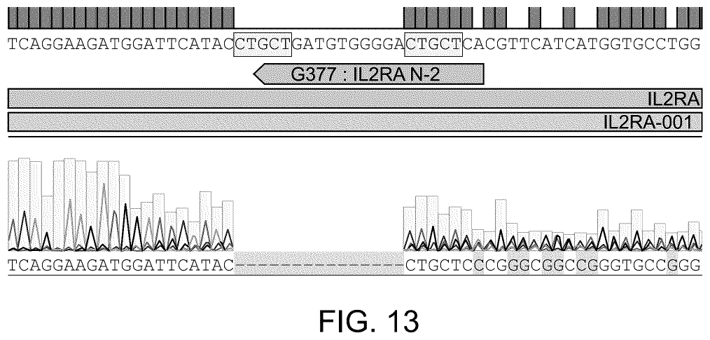

FIG. 13 shows amplicons analyzed by TIDE, from FIG. 12, aligned to human genome assembly GRCh38. Pictured are alignments of a sample treated with growth media, IL-2, M3814, and TSA for 24 hours after being electroporated with RNPs targeting IL2RA. As indicated from the indel spectrums in FIG. 3, a fifteen base-pair deletion was detected in the alignment. 5 bases of microhomology flank the deletion (highlighted in yellow) consistent with an MMEJ repair outcome.

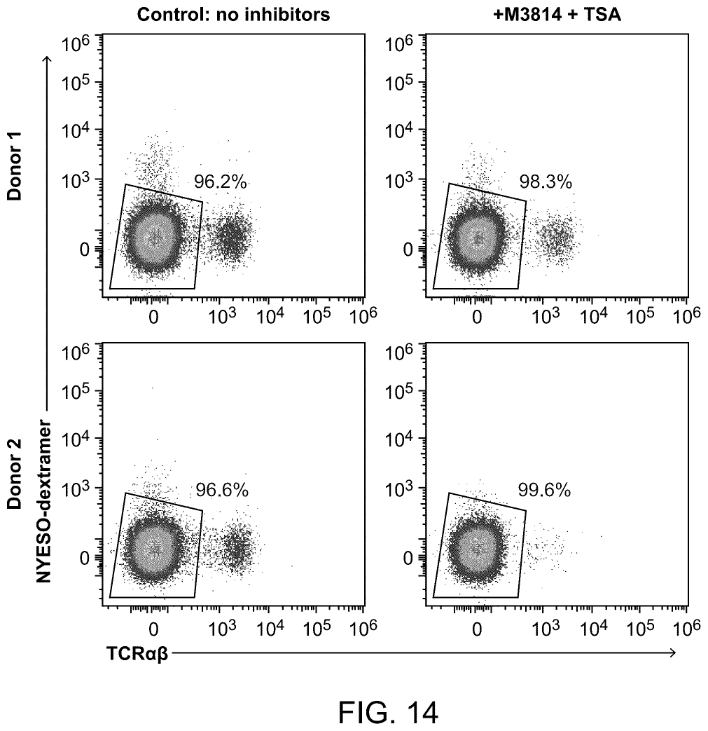

FIG. 14 shows the results of T cells from two independent healthy donors that were electroporated with RNPs targeting the TRAC locus. Following electroporations, cells were either grown in media and IL-2 or media, IL-2, M3814, and TSA for twenty-four hours. After twenty-four hours, media was removed and fresh IL-2 and growth media was added to all samples. Cells were collected five days after electroporations, stained with fluorescent antibodies for phenotyping, and analyzed by flow cytometry. The samples treated with inhibitors increase T cell receptor knockout, reaching up to 99.6% knockout.

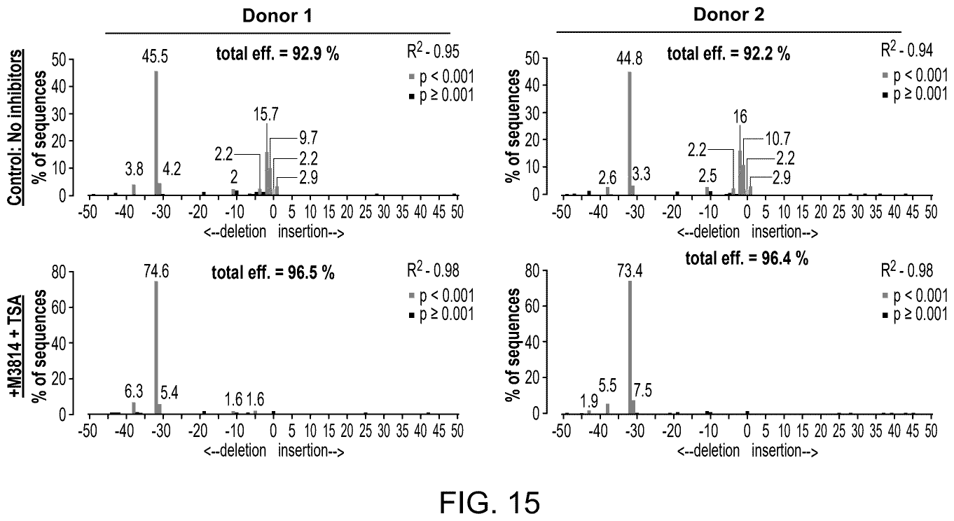

FIG. 15 shows the results of T cells from two independent, healthy donors that were electroporated with RNPs targeting the TRAC locus, and grown for twenty-four hours in either media and IL-2 or media, IL-2, M3814, and TSA as indicated. After twenty-four hours, media was removed and cells were resuspended in fresh media and IL-2. Four days after electroporations, cells were collected and genomic DNA was extracted from each sample prior to the generation of PCR amplicons containing the edited site. Amplicons were sequenced and then analyzed with TIDE to generate indel spectrums. Samples treated without inhibitors demonstrate a mix of small indels (characteristic of NHEJ) and a large 32 base pair deletion (characteristic of MMEJ). Samples that were treated with inhibitors displayed no small indels and, instead, almost entirely displayed 32 base pair deletions that can be attributed to MMEJ repair pathways.

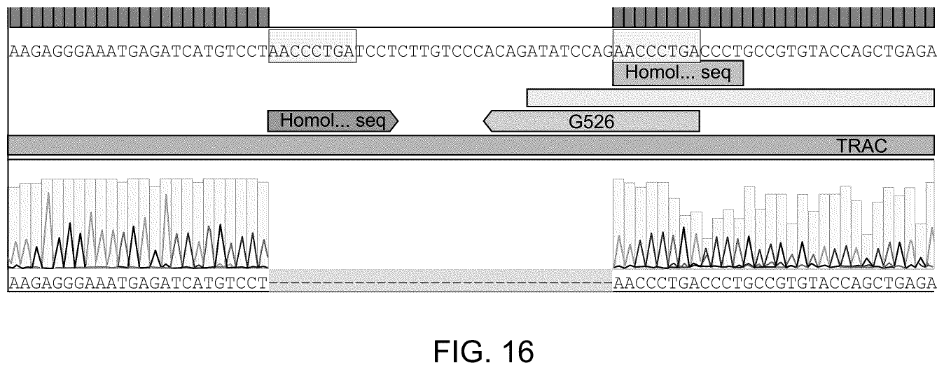

FIG. 16 shows the sequence of samples analyzed by TIDE, from FIG. 15, aligned to the human genome assembly GRCh38. Pictured is an alignment of a sample treated growth media, IL-2, M3814, and TSA for 24 hours following electroporation. As indicated in the indel spectrums in FIG. 5, a 32 base pair deletion is evident, and this deleted region is flanked by homologous 8 base pair sequences, consistent with dominant MMEJ repair.

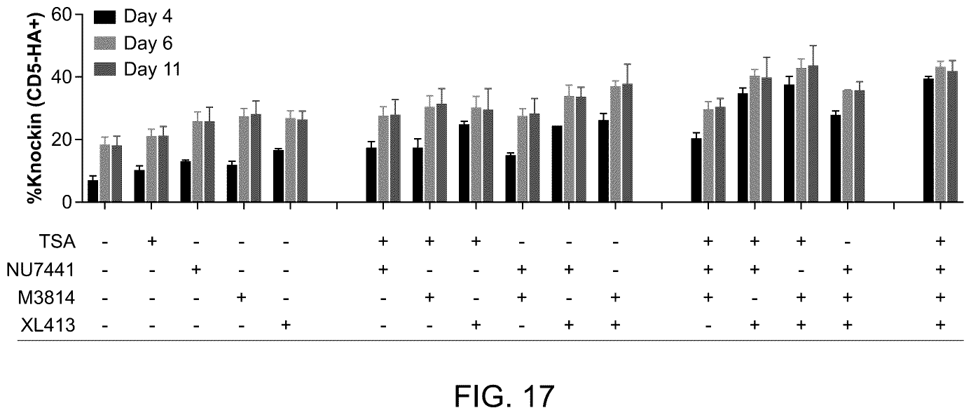

FIG. 17 shows the results of T cells from two independent, healthy donors that were electroporated with RNPs and ssODN HDRT encoding a HA-Tag targeted to the N-terminus of CD5. Cells were then treated with the indicated combinations of each inhibitor in growth media and IL-2. After twenty-four hours, media was removed from all samples and fresh media and IL-2 was added. At the indicated time points after electroporations (four, six, or eleven days), cells were collected, stained with fluorescent antibodies, and analyzed by flow cytometry for phenotyping and knock-in quantification. We found that each chemical not only increased HDR efficiencies (both when used alone and in combinations) but also accelerated the kinetics of HDR events. The combined usage of all four molecules allowed for knock-in efficiencies to reach maximal levels (measured by HA-Tag expression) four days after electroporations while samples not treated with inhibitors reached maximal levels six days after electroporations.

Altogether, the data demonstrate that desirable DNA repair outcomes in various gene editing scenarios can be directed using small molecules alone or in combination. Specific benefits include increased HDR integration efficiencies with the combinations of M3814, NU7441, TSA, and XL413, enhanced protein knockout efficiencies, and selectively leveraging MMEJ repair pathways to induce large deletion that may enable greater ease when dissecting both coding and non-coding regions of DNA in genetic screens.

### Sequences

HDRT sequence sequences are provided below, directly followed, is used for the HDRT, by 5′ CTS RC and 3′ CTS RC oligonucleotide sequences. “NA” means no CTS oligonucleotide was used for the specified HDRT. The first sequence listed after the HDRT sequence is the 5′ CTS RC oligonucleotide and the second sequence after the HDRT sequence is the 3′ CTS RC oligonucleotide sequence.

Lengthy table referenced here
US20250290099A1-20250918-T00001
Please refer to the end of the specification for access instructions.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

LENGTHY TABLES
The patent application contains a lengthy table section. A copy of the table is available in electronic form from the USPTO web site (https://seqdata.uspto.gov/?pageRequest=docDetail&DocID=US20250290099A1). An electronic copy of the table will also be available from the USPTO upon request and payment of the fee set forth in 37 CFR 1.19(b)(3).

1. A method of editing the genome of a cell, the method comprising inserting a nucleic acid sequence of an exogenous partial open reading frame (ORF) of an autosomal dominant gene into an intronic target region of an endogenous autosomal dominant gene in the cell, wherein:
the endogenous autosomal dominant gene comprises one or more disease-causing mutations,
the exogenous partial ORF of the autosomal dominant gene is free of disease-causing mutations, and
insertion of the exogenous partial ORF of the autosomal dominant gene into the intronic target region results in a modified autosomal dominant gene that encodes a protein which is free of disease-causing mutations.

2. The method of claim 1, wherein the autosomal dominant gene is CTLA4, and wherein insertion of an exogenous CLTA4 partial ORF into the intronic target region of an endogenous CLTA4 gene results in a modified CTLA4 gene that encodes a CTLA4 protein which is free of disease-causing mutations.

3. The method of claim 2, wherein the intronic target region is in intron 1 of the endogenous CTLA4 gene and the exogenous CTLA4 partial ORF comprises exons 2-4 of CTLA4.

4. (canceled)

5. The method of claim 2, wherein the nucleic acid sequence of the exogenous CTLA4 partial ORF is inserted into the intronic target region by introducing into the cell: (a) a targeted nuclease that creates an insertion site in the intronic target region; (b) a guide RNA that specifically hybridizes to the intronic target region; and (c) a DNA template comprising the nucleic acid sequence of the exogenous CTLA4 partial ORF.

6. The method of claim 5, wherein:
the DNA template is a single-stranded DNA template,
the 5′ end and the 3′ end of the DNA template comprise nucleotide sequences that are homologous to genomic sequences flanking the intronic target region,
the DNA template further comprises a nuclease binding sequence, wherein the nuclease binding sequence forms a double-stranded duplex with a complementary nucleotide sequence.

7. The method of claim 6, wherein the double-stranded duplex is formed with an oligonucleotide or polynucleotide comprising the complementary nucleotide sequence.

8. The method of claim 5, wherein the targeted nuclease is a Cas9 nuclease.

9. The method of claim 5, wherein the targeted nuclease, the guide RNA, and the DNA template are introduced into the cell as a ribonucleoprotein complex (RNP)-DNA template complex.

10. (canceled)

11. The method of claim 5, wherein the targeted nuclease, the guide RNA, and the DNA template are introduced into the cell in the presence of one or more small molecules selected from the group consisting of a DNA-dependent protein kinase (DNA-PK) inhibitor, a histone deacetylase (HDAC) inhibitor, and a cell division cycle 7-related protein kinase (CDC7) inhibitor.

12-14. (canceled)

15. The method of claim 1, further comprising administering the cell comprising the modified autosomal dominant gene to a human subject.

16. The method of claim 15, wherein the subject is same subject from whom the cell having the endogenous autosomal dominant gene was obtained.

17. (canceled)

18. The method of claim 1, wherein the cell is a T cell or a hematopoietic stem cell.

19. An isolated cell having an edited genome, which is prepared according to the method of claim 1.

20. An isolated cell having an edited genome comprising a modified CTLA4 gene comprising an CTLA4 open reading frame (ORF) comprising an endogenous exon 1 and exogenous exons 2-4, wherein the exogenous exons are free of disease-causing mutations.

21. (canceled)

22. The isolated cell of claim 20, which is a T cell or a hematopoietic stem cell.

23. A method for treating a haploinsufficiency, the method comprising administering a therapeutically effective amount of cells according to claim 19 to a subject in need thereof.

24. The method of claim 23, wherein the haploinsufficiency causes a primary immunodeficiency.

25. (canceled)

26. (canceled)

27. A method for modifying a target gene in a cell, the method comprising:
electroporating the cell in the presence of:
(a) a ribonucleoprotein (RNP) complex comprising a guide RNA and a targeted nuclease, wherein the guide RNA specifically hybridizes to a nucleotide sequence in a genomic target region and the targeted nuclease creates an insertion site in the genomic target region;
(b) a single-stranded DNA template comprising an exogenous nucleic acid sequence, wherein the 5′ end and the 3′ end of the DNA template comprise nucleotide sequences that are homologous to genomic sequences flanking the genomic target region, and wherein the DNA template further comprises a nuclease binding sequence, wherein the nuclease binding sequence forms a double-stranded duplex with a complementary nucleotide sequence; and
(c) one or more molecules selected from the group consisting of a DNA-dependent protein kinase (DNA-PK) inhibitor, a histone deacetylase (HDAC) inhibitor, and a cell division cycle 7-related protein kinase (CDC7) inhibitor,
thereby modifying the target gene.

28. The method of claim 27, wherein the DNA-PK inhibitor is (S)-(2-chloro-4-fluoro-5-(7-morpholinoquinazolin-4-yl)phenyl)(6-methoxypyridazin-3-yl)methanol (M3814) or 8-(dibenzo[b,d]thiophen-4-yl)-2-morpholino-4H-chromen-4-one (NU7441); and/or
the HDAC inhibitor is [R-(E,E)]-7-[4-(dimethylamino)phenyl]-N-hydroxy-4,6-dimethyl-7-oxo-2,4-heptadienamide (trichostatin A); and/or
the CDC7 inhibitor is (S)-8-chloro-2-(pyrrolidin-2-yl)benzofuro[3,2-d]pyrimidin-4(3H)-one hydrochloride (XL413).

29-34. (canceled)

35. The method of claim 27, wherein the human cell is a T cell or a hematopoietic stem cell.
